(19) United States
(12) Patent Application Publication
(10) Pub. No.: US 2007/0105862 A1
(43) Pub. Date: May 10, 2007

(54) Heat-shock protein binders

(76) Inventors: Milan Bruncko, Green Oaks, IL (US); Steven W. Elmore, Northbrook, IL (US); Xiaohong Song, Grayslake, IL (US); David J. Madar, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/593,315

(22) Filed: Nov. 6, 2006

Related U.S. Application Data
(60) Provisional application No. 60/735,716, filed on Nov. 10, 2005.

(51) Int. Cl. A61K 31/53; A61K 31/497; A61K 31/506; A61K 31/501; A61K 31/4439; A61K 31/427; C07D 417/02; C07D 413/02; C07D 403/02

(52) U.S. Cl. 514/241; 514/256; 514/255.05; 514/341; 514/367; 514/375; 514/252.06; 544/238; 544/209; 544/333; 544/405; 546/270.1; 546/273.1; 546/275.1; 548/156

(57) ABSTRACT

Compounds which bind to and inhibit the activity of HSP90, compositions containing the compounds and methods of treating diseases that are caused or exascerbated by overexpression of HSP90 are disclosed.

This application claims priority to U.S. Provisional Application Ser. No. 60/735,716, Nov. 10, 2005.

### FIELD OF THE INVENTION

This invention pertains to compounds which bind to and inhibit the activity of HSP90, compositions containing the compounds and methods of treating diseases which are caused or exacerbated by overexpression of HSP90.

### BACKGROUND OF THE INVENTION

Heat shock protein-90 (HSP90) is a molecular chaperone that participates in the function, folding and stabilization of client proteins, such as HER-2, Raf-1, Akt, Polo-1 and Met that are involved in oncogenic processes. The disruption of binding of client proteins to HSP90 in the N-terminal ATP-ase pocket reduces of these oncogenic proteins and provides simultaneous attack on cancer cells' growth and survival. Heat shock protein is therefore an attractive target in cancer therapies.

### SUMMARY OF THE INVENTION

One embodiment of this invention, therefore, pertains to compounds that bind to and inhibit the activity of HSP90, the compounds having formula (I) formula (II), and formula (III) 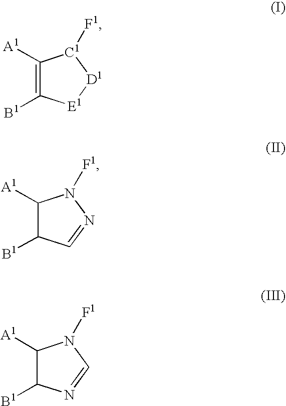
and therapeutically acceptable salts, prodrugs, salts of prodrugs and metabolites thereof, wherein

A¹ and B¹ are together and are benzene;

C¹ is C(H) or N;

D¹ is CH₂, C(O), NH, O, S, S(O) or SO₂ and E is CH₂ or NH, or

D¹ is CH₂ or NH and E¹ is CH₂, C(O), NH, O, S, S(O) or SO₂;

F¹ is phenol-2-yl which is unfused or fused with F^{1A} and substituted at the 4-position by OH, NH₂, NHR¹, N(R¹)₂, C(O)NH₂, C(O)NHR¹, C(O)N(R¹)₂, NHC(O)R¹, NR¹ C(O)R¹, SO₂NH₂, SO₂NHR¹, SO₂N(R¹)₂, NHSO₂R¹ or NR¹SO₂R¹;

F^{1A} is benzene, heteroarene, cycloalkane, cycloalkene, heterocycloalkane or heterocycloalkene;

R¹ is R², R³, R⁴ or R⁵;

R² is phenyl which is unfused or fused with benzene, heteroarene or R^{2A}; R^{2A} is cycloalkane, cycloalkene, heterocycloalkane or heterocycloalkene;

R³ is heteroaryl which is unfused or fused with benzene, heteroarene or R^{3A}; R^{3A i}s cycloalkane, cycloalkene, heterocycloalkane or heterocycloalkene;

R⁴ is cycloalkyl, cycloalkenyl, heterocycloalkyl or heterocycloalkenyl, each of which is unfused or fused with benzene, heteroarene or R^{4A}; R^{4A} is cycloalkane, cycloalkene, heterocycloalkane or heterocycloalkene;

R⁵ is alkyl, alkenyl or alkynyl, each of which is unsubstituted or substituted with one or two or three of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHSO₂R⁶, NR⁶SO₂R⁶, NHC(O)OR⁶, NR⁶C(O)OR⁶, SO₂NH₂, SO₂NHR⁶, SO₂N(R⁶)₂, NHC(O)NH₂, NHC(O)NHR⁶, NHC(O)N(R⁶)₂, NR⁶C(O)N(R⁶)₂, OH, (O), C(O)OH, CN, NH₂, CF₃, OCF₃, CF₂CF₃, F, Cl, Br or I;

R⁶ is R⁷, R⁸, R⁹ or R¹⁰;

R⁷ is phenyl which is unfused or fused with benzene, heteroarene or R^{7A}; R^{7A} is cycloalkane, cycloalkene, heterocycloalkane or heterocycloalkene;

R⁸ is heteroaryl which is unfused or fused with benzene, heteroarene or R^{8A}; R^{8A} is cycloalkane, cycloalkene, heterocycloalkane or heterocycloalkene;

R⁹ is cycloalkyl, cycloalkenyl, heterocycloalkyl or heterocycloalkenyl, each of which is unfused or fused with benzene, heteroarene or R^{9A}; R^{9A} is cycloalkane, cycloalkene, heterocycloalkane or heterocycloalkene;

R¹⁰ is alkyl, alkenyl or alkynyl, each of which is unsubstituted or substituted with one or two or three of independently selected OR¹¹, SR¹¹, S(O)R¹¹, SO₂R¹¹, NH₂, NHR¹¹, N(R¹¹)₂, C(O)R¹¹, C(O)NH₂, C(O)NHR¹¹, C(O)N(R¹¹)₂, NHC(O)R¹¹, NR¹¹C(O)R¹¹, NHSO₂R¹¹, NR¹¹SO₂R¹¹, NHC(O)OR¹¹, NR¹¹C(O)OR¹¹, SO₂NH₂, SO₂NHR¹¹, SO₂N(R¹¹)₂, NHC(O)NH₂, NHC(O)NHR¹¹, NHC(O)N(R¹¹)₂, NR¹¹C(O)N(R¹¹)₂, OH, (O), C(O)H, C(O)OH, CN, CF₃, OCF₃, CF₂CF₃, F, Cl, Br or I;

R¹¹ is alkyl, alkenyl, alkynyl, phenyl, naphthyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl;

wherein the benzene represented by A¹ and B¹ together and the moieties represented by F¹ and F^{1A} are independently unsubstituted or substituted or further substituted with one or two or three or four of independently selected R¹², OR¹², SR¹², S(O)R¹², SO₂R¹², NH₂, NHR¹², N(R¹²)₂, C(O)R¹², C(O)OR¹², C(O)NH₂, C(O)NHR¹², C(O)N(R¹²)₂, NHC(O)R¹², NR¹²C(O)R¹², NHSO₂R¹², NR¹²SO₂R¹², NHC(O)OR₁₂, NR¹²C(O)OR¹², SO₂NH₂, SO₂NHR¹², SO₂N(R¹²)₂, NHC(O)NH₂, NHC(O)NHR¹², NHC(O)N(R¹²)₂, NR¹²C(O)N(R¹²)₂, C(N)NH₂, C(N)NHR¹², C(N)N(R¹²)₂, NHC(N)NH₂, NHC(N)NHR¹², NHC(N)N(R¹²)₂, OH, (O), C(O)H, C(O)OH, NO₂, CN, CF₃, OCF₃, CF₂CF₃, F, Cl, Br or I;

wherein R¹² is R¹³, R¹⁴, R¹⁵ or R¹⁶;

R¹³ is phenyl which is unfused or fused with benzene, heteroarene or R^{13A}; R^{13A} is cycloalkane, cycloalkene, heterocycloalkane or heterocycloalkene;

R¹⁴ is heteroaryl which is unfused or fused with benzene, heteroarene or R^{4A}; RK^{4A} is cycloalkane, cycloalkene, heterocycloalkane or heterocycloalkene;

R¹⁵ is cycloalkyl, cycloalkenyl, heterocycloalkyl or heterocycloalkenyl, each of which is unfused or fused with benzene, heteroarene or R^{5A}; R^{5A} is cycloalkane, cycloalkene, heterocycloalkane or heterocycloalkene;

R¹⁶ is alkyl, alkenyl or alkynyl, each of which is unsubstituted or substituted with one or two or three of independently selected R¹⁷, OR¹⁷, SR¹⁷, S(O)R₁₇, SO₂R¹⁷, NH₂, NHR¹⁷, N(R¹⁷)₂, C(O)R¹⁷, C(O)NH₂, C(O)NHR¹⁷, C(O)N(R¹⁷)₂, NHC(O)R¹⁷, NR¹⁷C(O)R¹⁷, NHSO₂R¹⁷, NR¹⁷SO₂R¹⁷, NHC(O)OR¹⁷, NR¹⁷C(O)OR¹⁷, SO₂NH₂, SO₂NHR¹⁷, SO₂N(R¹⁷)₂, NHC(O)NH₂, NHC(O)NHR¹⁷, NHC(O)N(R¹⁷)₂, NR¹⁷C(O)N(R¹⁷)₂, C(N) NH₂, C(N)NHR¹⁷, OH, (O), C(O)H, C(O)OH, CN, CF₃, CF₂CF₃, OCF₃, F, Cl, Br or I;

R¹⁷ is R¹⁸, R²⁰, R²⁰ or R²¹;

R¹⁸ is phenyl which is unfused or fused with benzene, heteroarene or R^{18A}; R^{18A} is cycloalkane, cycloalkene, heterocycloalkane or heterocycloalkene;

R¹⁹ is heteroaryl which is unfused or fused with benzene, heteroarene or R^{19A}; R^{19A} is cycloalkane, cycloalkene, heterocycloalkane or heterocycloalkene;

R²⁰ is cycloalkyl, cycloalkenyl, heterocycloalkyl or heterocycloalkenyl, each of which is unfused or fused with benzene, heteroarene or R^{20A}; R^{20A} is cycloalkane, cycloalkene, heterocycloalkane or heterocycloalkene;

R²¹ is alkyl, alkenyl or alkynyl, each of which is unsubstituted or substituted with one or two or three of independently selected R²², OR²², SR²², S(O)R²², SO₂R²² , NH₂, NHR²², N(R²² )₂, C(O)R²², C(O)NH₂, C(O)NHR²², C(O)N(R²² )₂, NHC(O)R²², NR²²C(O)R²², NHSO₂R²², NR²²SO₂R²², NHC(O)OR²², NR¹⁷C(O)OR²², SO₂NH₂, SO₂NHR²², SO₂N(R²²)₂, NHC(O)NH₂, NHC(O)NHR²², NHC(O)N(R²²)₂, NR²²C(O)N(R²²)₂, OH, (O), C(O)H, C(O)OH, CN, CF₃, OCF₃, CF₂CF₃, F, Cl, Br or I;

R²² is R²³, R²⁴, R²⁵ or R²⁶;

R²³ is phenyl which is unfused or fused with benzene, heteroarene or R^{23A}; R^{23A} is cycloalkane, cycloalkene, heterocycloalkane or heterocycloalkene;

R²⁴ is heteroaryl which is unfused or fused with benzene, heteroarene or R^{24A}; R^{24A} is cycloalkane, cycloalkene, heterocycloalkane or heterocycloalkene;

R²⁵ is cycloalkyl, cycloalkenyl, heterocycloalkyl or heterocycloalkenyl, each of which is unfused or fused with benzene, heteroarene or R^{25A}; R^{25A} is cycloalkane, cycloalkene, heterocycloalkane or heterocycloalkene;

R²⁶ is alkyl, alkenyl or alkynyl;

wherein the moieties represented by R¹³, R¹⁴, R¹⁵, R¹⁸, R¹⁹ and R²⁰ are independently unsubstituted or substituted or further substituted with one or two or three or four of independently selected R²⁷, OR²⁷, SR²⁷, S(O)R²⁷, SO₂R²⁷, NH₂, NHR²⁷, N(R²⁷)₂, C(O)R²⁷, C(O)OR²⁷, C(O)NH₂, C(O)NHR²⁷, C(O)N(R²⁷)₂, NHC(O)R²⁷, NR²⁷C(O)R²⁷, NHSO₂R²⁷, NR²⁷ R₂, NHC(O)OR²⁷, NR²⁷ C(O)OR²⁷, SO₂NH₂, SO₂NHR²⁷, SO₂N(R²⁷)₂, NHC(O)NH₂, NHC(O)NHR²⁷ , NHC(O)N(R²⁷)₂, NR²⁷C(O)N(R²⁷)₂, C(N)NH₂, C(N)NHR²⁷, C(N)N(R²⁷)₂, NHC(N)NH₂, NHC(N)NHR²⁷, NHC(N)N(R²⁷)₂, OH, (O), C(O)H, C(O)OH, NO₂, CN, CF₃, OCF₃, CF₂CF₃, F, Cl, Br or I;

R²⁷ is R²⁸, R²⁹, R³⁰ or R³¹;

R²⁸ is phenyl which is unfused or fused with benzene, heteroarene or R^{27A}; R^{27A} is cycloalkane, cycloalkene, heterocycloalkane or heterocycloalkene;

R²⁹ is heteroaryl which is unfused or fused with benzene, heteroarene or R^{28A}; R^{28A} is cycloalkane, cycloalkene, heterocycloalkane or heterocycloalkene;

R³⁰ is cycloalkyl, cycloalkenyl, heterocycloalkyl or heterocycloalkenyl, each of which is unfused or fused with one or two of independently selected benzene, heteroarene or R^{29A} R^{29A} is cycloalkane, cycloalkene, heterocycloalkane or heterocycloalkene;

R³¹ is alkyl, alkenyl or alkynyl;

wherein the moieties represented by R²⁸, R²⁹ and R³⁰ are unsubstituted or substituted with OH, (O), C(O)H, C(O)OH, NO₂, CN, CF₃, OCF₃, CF₂CF₃, F, Cl, Br or I.

Still another embodiment pertains to compositions for treating diseases during which are expressed HSP90, said compositions comprising an excipient and a therapeutically effective amount of a compound having formula (I), formula (II) or formula (III).

Still another embodiment pertains to methods of treating diseases in a patient during which are expressed HSP90, said methods comprising administering to the patient a therapeutically effective amount of a compound having formula (I), formula (II) or formula (III).

Still another embodiment pertains to compositions for treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma, said compositions comprising an excipient and a therapeutically effective amount of the compound having formula (I), formula (II) or formula (III).

Still another embodiment pertains to methods of treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma, said methods comprising administering to the patient a therapeutically effective amount of a compound having formula (I), formula (II) or formula (III).

Still another embodiment pertains to compositions for treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma in a patient during which is expressed HSP90, said compositions comprising an excipient and a therapeutically effective amount of the compound having formula (I), formula (II) or formula (III) and a therapeutically effective amount of one additional therapeutic agent or more than one additional therapeutic agent.

Still another embodiment pertains to methods of treating diseases in a patient during which is expressed HSP90, said methods comprising administering to the patient a therapeutically effective amount of a compound having formula (I), formula (II) or formula (III) and a therapeutically effective amount of one additional therapeutic agent or more than one additional therapeutic agent.

Still another embodiment pertains to compositions for treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma, said compositions comprising an excipient and a therapeutically effective amount of the compound having formula (I), formula (II) or formula (III) and a therapeutically effective amount of one additional therapeutic agent or more than one additional therapeutic agent.

Still another embodiment pertains to methods of treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma, said methods comprising administering to the patient a therapeutically effective amount of the compound having formula (I), formula (II) or formula (III) and a therapeutically effective amount of one additional therapeutic agent or more than one additional therapeutic agent.

Still another embodiment pertains to the compounds

1-(5-chloro-2,4-dihydroxyphenyl)-5-(trifluoromethyl)-1,3-dihydro-2H-benzimidazol-2-one, 1-(2,4-dihydroxyphenyl)-5-(trifluoromethyl)-1,3-dihydro-2H-benzimidazol-2-one,

1-(5-chloro-2,4-dihydroxyphenyl)-1,3-dihydro-2H-benzimidazol-2-one,

1-(2,4-dihydroxyphenyl)-1,3-dihydro-2H-benzimidazol-2-one, 1-(5-bromo-2,4-dihydroxyphenyl)-5-(trifluoromethyl)-1,3-dihydro-2H-benzimidazol-2-one,

1-(5-chloro-2,4-dihydroxyphenyl)-7-fluoro-5-(trifluoromethyl)-1,3-dihydro-2H-benzimidazol-2-one,

1-(5-chloro-2,4-dihydroxyphenyl)-2-oxo-2,3-dihydro-1H-benzimidazole-5-carbonitrile,

3-(5-chloro-2,4-dihydroxyphenyl)-2-oxo-2,3-dihydro-1H-benzimidazole-5-carbonitrile,

1-(5-chloro-2,4-dihydroxyphenyl)-5-fluoro-1,3-dihydro-2H-benzimidazol-2-one,

1-(5-chloro-2,4-dihydroxyphenyl)-2-oxo-2,3-dihydro-1H-benzimidazole-5-sulfonamide,

1-(5-chloro-2,4-dihydroxyphenyl)-5-nitro-1,3-dihydro-2H-benzimidazol-2-one,

5-chloro-1-(5-chloro-2,4-dihydroxyphenyl)-1,3-dihydro-2H-benzimidazol-2-one,

5-bromo-1-(5-chloro-2,4-dihydroxyphenyl)-1,3-dihydro-2H-benzimidazol-2-one,

1-(4,6-dihydroxy-1,1′-biphenyl-3-yl)-5-(trifluoromethyl)-1,3-dihydro-2H-benzimidazol-2-one,

1-(5-bromo-2,4-dihydroxyphenyl)-1,3-dihydro-2H-benzimidazol-2-one, 1-(2,4-dihydroxy-5-((E)-2-phenylvinyl)phenyl)-5-(trifluoromethyl)-1,3-dihydro-2H-benzimidazol-2-one,

1-(2,4-dihydroxy-5-((E)-2-phenylvinyl)phenyl)-1,3-dihydro-2H-benzimidazol-2-one,

1-(4,6-dihydroxy-1,1′-biphenyl-3-yl)-1,3-dihydro-2H-benzimidazol-2-one, 1-(2′,4,6-trihydroxy-1,1′-biphenyl-3-yl)-1,3-dihydro-2H-benzimidazol-2-one,

1-(4′-chloro-4,6-dihydroxy-1,1′-biphenyl-3-yl)-1,3-dihydro-2H-benzimidazol-2-one,

1-(2′-chloro-4,6-dihydroxy-1,1′-biphenyl-3-yl)-1,3-dihydro-2H-benzimidazol-2-one,

1-(3′-chloro-4,6-dihydroxy-1,1′-biphenyl-3-yl)-1,3-dihydro-2H-benzimidazol-2-one,

1-(2,4-dihydroxy-5-propylphenyl)-1,3-dihydro-2H-benzimidazol-2-one, 1-(5-chloro-2,4-dihydroxyphenyl)-5-(4-hydroxyphenyl)-1,3-dihydro-2H-benzimidazol-2-one,

1-(3′-chloro-4,6-dihydroxy-1,1′-biphenyl-3-yl)-1,3-dihydro-2H-benzimidazol-2-one,

methyl 5-(7-chloro-2-oxo-5-(trifluoromethyl)-2,3-dihydro-1H-benzimidazol-1-yl)-4-hydroxy-2-methoxybenzoate,

methyl 1-(5-bromo-2,4-dihydroxyphenyl)-2-oxo-2,3-dihydro-1H-benzimidazole-5-carboxylate,

1-(2,4-dihydroxy-5-(2-phenylethyl)phenyl)-1,3-dihydro-2H-benzimidazol-2-one,

1-(2′-fluoro-4,6-dihydroxy-5′-methyl-1,1′-biphenyl-3-yl)-5-(trifluoromethyl)-1,3-dihydro-2H-benzimidazol-2-one,

1-(3′-chloro-4,6-dihydroxy-4′-methyl-1,1′-biphenyl-3-yl)-5-(trifluoromethyl)-1,3-dihydro-2H-benzimidazol-2-one,

5-(trifluoromethyl)-1-(2′,4,6-trihydroxy-5′-isopropyl-1,1′-biphenyl-3-yl)-1,3-dihydro-2H-benzimidazol-2-one,

1-(2′-fluoro-4,6-dihydroxy-5′-(trifluoromethyl)-1,1′-biphenyl-3-yl)-5-(trifluoromethyl)-1,3-dihydro-2H-benzimidazol-2-one,

1-(3′-fluoro-4,6-dihydroxy-1,1′:4′,1″-terphenyl-3-yl)-5-(trifluoromethyl)-1,3-dihydro-2H-benzimidazol-2-one, 1-(4′-chloro-4,6-dihydroxy-3′-(trifluoromethyl)-1,1′-biphenyl-3-yl)-5-(trifluoromethyl)-1,3-dihydro-2H-benzimidazol-2-one,

1-(4′-benzoyl-4,6-dihydroxy-1,1′-biphenyl-3-yl)-5-(trifluoromethyl)-1,3-dihydro-2H-benzimidazol-2-one,

N-(tert-butyl)-2′,4′-dihydroxy-5′-(2-oxo-5-(trifluoromethyl)-2,3-dihydro-1H-benzimidazol-1-yl)-1,1′-biphenyl-3-carboxamide,

N-cyclopentyl-2′,4′-dihydroxy-5′-(2-oxo-5-(trifluoromethyl)-2,3-dihydro-1H-benzimidazol-1-yl)-1,1′-biphenyl-3-carboxamide,

N-ethyl-2′,4′-dihydroxy-5′-(2-oxo-5-(trifluoromethyl)-2,3-dihydro-1H-benzimidazol-1-yl)-1,1′-biphenyl-3-carboxamide,

N-cyclohexyl-2′,4′-dihydroxy-5′-(2-oxo-5-(trifluoromethyl)-2,3-dihydro-1H-benzimidazol-1-yl)-1,1′-biphenyl-3-carboxamide,

1-(5-(2-cyclohexylethyl)-2,4-dihydroxyphenyl)-1,3-dihydro-2H-benzimidazol-2-one,

5-bromo-1-(5-bromo-2,4-dihydroxyphenyl)-1,3-dihydro-2H-benzimidazol-2-one,

6-bromo-1-(5-bromo-2,4-dihydroxyphenyl)-1,3-dihydro-2H-benzimidazol-2-one,

6-bromo-1-(5-chloro-2,4-dihydroxyphenyl)-1,3-dihydro-2H-benzimidazol-2-one,

1-(5-chloro-2,4-dihydroxyphenyl)-6-phenyl-1,3-dihydro-2H-benzimidazol-2-one,

1-(4′-acetyl-4,6-dihydroxy-1,1′-biphenyl-3-yl)-5-(trifluoromethyl)-1,3-dihydro-2H-benzimidazol-2-one,

1-(4,6-dihydroxy-2′,3′-dimethyl-1,1′-biphenyl-3-yl)-5-(trifluoromethyl)-1,3-dihydro-2H-benzimidazol-2-one,

1-(4,6-dihydroxy-4′-(trifluoromethoxy)-1,1′-biphenyl-3-yl)-5-(trifluoromethyl)-1,3-dihydro-2H-benzimidazol-2-one,

1-(4,6-dihydroxy-2′,5′-dimethyl-1,1′-biphenyl-3-yl)-5-(trifluoromethyl)-1,3-dihydro-2H-benzimidazol-2-one,

1-(4,6-dihydroxy-3′,5′-dimethyl-1,1′-biphenyl-3-yl)-5-(trifluoromethyl)-1,3-dihydro-2H-benzimidazol-2-one,

N-(2′,4′-dihydroxy-5′-(2-oxo-5-(trifluoromethyl)-2,3-dihydro-1H-benzimidazol-1-yl)-1,1′-biphenyl-3-yl)acetamide,

1-(2′,3′-dichloro-4,6-dihydroxy-1,1′-biphenyl-3-yl)-5-(trifluoromethyl)-1,3-dihydro-2H-benzimidazol-2-one,

1-(2′,4′-dichloro-4,6-dihydroxy-1,1′-biphenyl-3-yl)-5-(trifluoromethyl)-1,3-dihydro-2H-benzimidazol-2-one,

1-(2′,5′-dichloro-4,6-dihydroxy-1,1′-biphenyl-3-yl)-5-(trifluoromethyl)-1,3-dihydro-2H-benzimidazol-2-one,

1-(4,6-dihydroxy-2′-methyl-1,1′-biphenyl-3-yl)-5-(trifluoromethyl)-1,3-dihydro-2H-benzimidazol-2-one,

1-(4,6-dihydroxy-4′-methyl-1,1′-biphenyl-3-yl)-5-(trifluoromethyl)-1,3-dihydro-2H-benzimidazol-2-one,

1-(3′-fluoro-4,6-dihydroxy-1,1′-biphenyl-3-yl)-5-(trifluoromethyl)-1,3-dihydro-2H-benzimidazol-2-one,

1-(4′-fluoro-4,6-dihydroxy-1,1′-biphenyl-3-yl)-5-(trifluoromethyl)-1,3-dihydro-2H-benzimidazol-2-one,

1-(2′-chloro-4,6-dihydroxy-1,1′-biphenyl-3-yl)-5-(trifluoromethyl)-1,3-dihydro-2H-benzimidazol-2-one,

1-(3′-chloro-4,6-dihydroxy-1,1′-biphenyl-3-yl)-5-(trifluoromethyl)-1,3-dihydro-2H-benzimidazol-2-one,

1-(4′-chloro-4,6-dihydroxy-1,1′-biphenyl-3-yl)-5-(trifluoromethyl)-1,3-dihydro-2H-benzimidazol-2-one,

1-(4,6-dihydroxy-3′-(trifluoromethyl)-1,1′-biphenyl-3-yl)-5-(trifluoromethyl)-1,3-dihydro-2H-benzimidazol-2-one,

1-(2,4-dihydroxy-5-(naphth-1-yl)phenyl)-1,3-dihydro-2H-benzimidazol-2-one,

1-(2,4-dihydroxy-5-(naphth-2-yl)phenyl)-1,3-dihydro-2H-benzimidazol-2-one,

1-(2,4-dihydroxy-5-quinolin-8-ylphenyl)-1,3-dihydro-2H-benzimidazol-2-one,

2,4-dihydroxy-5-quinolin-4-ylphenyl)-1,3-dihydro-2H-benzimidazol-2-one,

1-(4,6-dihydroxy-3′,4′,5′-trimethoxy-1,1′-biphenyl-3-yl)-1,3-dihydro-2H-benzimidazol-2-one,

1-(4,6-dihydroxy-1,1′:3′,1″-terphenyl-3-yl)-1,3-dihydro-2H-benzimidazol-2-one,

1-(4,6-dihydroxy-1,1′:4′,1″-terphenyl-3-yl)-1,3-dihydro-2H-benzimidazol-2-one,

1-(3′,5′-dichloro-4,6-dihydroxy-1,1′-biphenyl-3-yl)-1,3-dihydro-2H-benzimidazol-2-one,

1-(3′,4′-dichloro-4,6-dihydroxy-1,1′-biphenyl-3-yl)-1,3-dihydro-2H-benzimidazol-2-one,

1-(4,6-dihydroxy-2′-methyl-1,1′-biphenyl-3-yl)-1,3-dihydro-2H-benzimidazol-2-one,

1-(2,4-dihydroxy-5-((E)-2-(4-methylphenyl)vinyl)phenyl)-1,3-dihydro-2H-benzimidazol-2-one,

1-(5-((E)-2-(3-fluorophenyl)vinyl)-2,4-dihydroxyphenyl)-1,3-dihydro-2H-benzimidazol-2-one,

1-(5-((E)-2-(4-fluorophenyl)vinyl)-2,4-dihydroxyphenyl)-1,3-dihydro-2H-benzimidazol-2-one,

1-(5-((E)-2-(4-chlorophenyl)vinyl)-2,4-dihydroxyphenyl)-1,3-dihydro-2H-benzimidazol-2-one,

2,4-dihydroxy-5-(2-oxo-2,3-dihydro-1H-benzimidazol-1-yl)benzonitrile

1-(3′-acetyl-4,6-dihydroxy-1,1′-biphenyl-3-yl)-5-(trifluoromethyl)-1,3-dihydro-2H-benzimidazol-2-one,

6-(aminomethyl)-1-(5-chloro-2,4-dihydroxyphenyl)-1,3-dihydro-2H-benzimidazol-2-one trifluoroacetate,

1-(2,4-dihydroxy-5-(2-(4-methylphenyl)ethyl)phenyl)-1,3-dihydro-2H-benzimidazol-2-one,

1-(2,4-dihydroxy-5-thien-3-ylphenyl)-1,3-dihydro-2H-benzimidazol-2-one,

1-(5-(2-(4-fluorophenyl)ethyl)-2,4-dihydroxyphenyl)-1,3-dihydro-2H-benzimidazol-2-one,

1-(2,4-dihydroxy-5-((E)-2-(4-(trifluoromethyl)phenyl)vinyl)phenyl)-1,3-dihydro-2H-benzimidazol-2-one,

1-(2′,4′-dichloro-4,6-dihydroxy-1,1′-biphenyl-3-yl)-1,3-dihydro-2H-benzimidazol-2-one,

1-(4,6-dihydroxy-4′-methyl-1,1′-biphenyl-3-yl)-1,3-dihydro-2H-benzimidazol-2-one,

1-(4,6-dihydroxy-2′-phenoxy-1,1′-biphenyl-3-yl)-1,3-dihydro-2H-benzimidazol-2-one,

1-(5-((1E)-3 ,3-dimethylbut-1-enyl)-2,4-dihydroxyphenyl)-1,3-dihydro-2H-benzimidazol-2-one,

1-(5-(2-(3-fluorophenyl)ethyl)-2,4-dihydroxyphenyl)-1,3-dihydro-2H-benzimidazol-2-one,

1-(5-((E)-2-cyclohexylvinyl)-2,4-dihydroxyphenyl)-1,3-dihydro-2H-benzimidazol-2-one,

1-(4,6-dihydroxy-3′-(trifluoromethoxy)-1,1′-biphenyl-3-yl)-5-(trifluoromethyl)-1,3-dihydro-2H-benzimidazol-2-one,

1-(2′,5′-dichloro-4,6-dihydroxy-1,1′-biphenyl-3-yl)-1,3-dihydro-2H-benzimidazol-2-one,

1-(2′,3′-dichloro-4,6-dihydroxy-1,1′-biphenyl-3-yl)-1,3-dihydro-2H-benzimidazol-2-one,

1-(4,6-dihydroxy-2′-(trifluoromethyl)-1,1′-biphenyl-3-yl)-1,3-dihydro-2H-benzimidazol-2-one,

1-(4,6-dihydroxy-2′-isopropyl-1,1′-biphenyl-3-yl)-1,3-dihydro-2H-benzimidazol-2-one,

1-(2,4-dihydroxy-5-((E)-2-(4-methoxyphenyl)vinyl)phenyl)-1,3-dihydro-2H-benzimidazol-2-one,

1-(4,6-dihydroxy-2′-methoxy-1,1′-biphenyl-3-yl)-1,3-dihydro-2H-benzimidazol-2-one,

5-(aminomethyl)-1-(5-chloro-2,4-dihydroxyphenyl)-1,3-dihydro-2H-benzimidazol-2-one trifluoroacetate,

1-(5-(3,3-dimethylbutyl)-2,4-dihydroxyphenyl)-1,3-dihydro-2H-benzimidazol-2-one,

1-(2,4-dihydroxy-5-(2-(4-methoxyphenyl)ethyl)phenyl)-1,3-dihydro-2H-benzimidazol-2-one,

1-(5-(2-(1,1′-biphenyl-4-yl)ethyl)-2,4-dihydroxyphenyl)-1,3-dihydro-2H-benzimidazol-2-one,

N-((1-(5-chloro-2,4-dihydroxyphenyl)-2-oxo-2,3-dihydro-1H-benzimidazol-5-yl)methyl)acetamide,

N-((1-(5-chloro-2,4-dihydroxyphenyl)-2-oxo-2,3-dihydro-1H-benzimidazol-5-yl)methyl)benzamide,

N-((1-(5-chloro-2,4-dihydroxyphenyl)-2-oxo-2,3-dihydro-1H-benzimidazol-5-yl)methyl)methanesulfonamide,

N-((1-(5-chloro-2,4-dihydroxyphenyl)-2-oxo-2,3-dihydro-1H-benzimidazol-5-yl)methyl)-4-methylbenzenesulfonamide,

N-((1-(5-chloro-2,4-dihydroxyphenyl)-2-oxo-2,3-dihydro-1H-benzimidazol-5-yl)methyl)-N′-phenylurea,

N-((1-(5-chloro-2,4-dihydroxyphenyl)-2-oxo-2,3-dihydro-1H-benzimidazol-5-yl)methyl)-N′-ethylurea,

1-(2,4-dihydroxy-5-(2-hydroxypyridin-3-yl)phenyl)- I ,3-dihydro-2H-benzimidazol-2-one,

1-(2,4-dihydroxy-5-(2-hydroxypyridin-3-yl)phenyl)-1,3-dihydro-2H-benzimidazol-2-one,

1-(2,4-dihydroxy-5-(4-methylthien-3-yl)phenyl)-1,3-dihydro-2H-benzimidazol-2-one,

1-(2′-ethyl-4,6-dihydroxy-1,1′-biphenyl-3-yl)-1,3-dihydro-2H-benzimidazol-2-one,

1-(5-(1,2-dimethylprop-1-enyl)-2,4-dihydroxyphenyl)-1,3-dihydro-2H-benzimidazol-2-one,

1-(6-hydroxy-2,2,3-trimethyl-2,3-dihydro-1-benzofuran-5-yl)-1,3-dihydro-2H-benzimidazol-2-one,

1-(2,4-dihydroxy-5-(2-(4-(trifluoromethyl)phenyl)ethyl)phenyl)-3,5-dihydrocyclopenta(d)imidazol-2(1 H)-one,

1-(5-cyclopentyl-2,4-dihydroxyphenyl)-1,3-dihydro-2H-benzimidazol-2-one,

1-(5-cyclohexyl-2,4-dihydroxyphenyl)-1,3-dihydro-2H-benzimidazol-2-one,

1-(2,4-dihydroxy-5-(3-phenylpropyl)phenyl)-1,3-dihydro-2H-benzimidazol-2-one,

5-((((3-(5-chloro-2,4-dihydroxyphenyl)-2-oxo-2,3-dihydro-1H-benzimidazol-5-yl)methyl)amino)carbonyl)-2-(6-hydroxy-3-oxo-3H-xanthen-9-yl)benzoic acid,

5-((((1-(5-chloro-2,4-dihydroxyphenyl)-2-oxo-2,3-dihydro-1H-benzimidazol-5-yl)methyl)amino)carbonyl)-2-(6-hydroxy-3-oxo-3H-xanthen-9-yl)benzoic acid and

5-((((1-(5-chloro-2,4-dihydroxyphenyl)-2-oxo-2,3-dihydro-1H-benzimidazol-5-yl)methyl)amino)carbonyl)-2-(6-hydroxy-3-oxo-3H-xanthen-9-yl)benzoic acid, and therapeutically acceptable salts, prodrugs, salts of prodrugs and metabolites thereof.

### DETAILED DESCRIPTION OF THE INVENTION

Variable moieties of compounds herein are represented by identifiers (capital letters with numerical and/or alphabetical superscripts) and may be specifically embodied.

It is meant to be understood that proper valences are maintained for all moieties and combinations thereof, that monovalent moieties having more than one atom are attached through their left ends.

It is also meant to be understood that a specific embodiment of a variable moiety may be the same or different as another specific embodiment having the same identifier.

The term “cycloalkane,” as used herein, means C₃-cycloalkane, C₄-cycloalkane, C₅-cycloalkane and C₆-cycloalkane.

The term “cycloalkyl,” as used herein, means C₃-cycloalkyl, C₄-cycloalkyl, C₅-cycloalkyl and C₆-cycloalkyl.

The term “cycloalkene,” as used herein, means C₄-cycloalkene, C₅-cycloalkene and C₆-cycloalkene.

The term “cycloalkenyl,” as used herein, means C₄-cycloalkenyl, C₅-cycloalkenyl and C₆-cycloalkenyl.

The term “heteroarene,” as used herein, means furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine and 1,2,3-triazole.

The term “heteroaryl,” as used herein, means furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl and 1,2,3-triazolyl.

The term “heterocycloalkane,” as used herein, means cycloalkane having one or two or three CH₂ moieties replaced with independently selected O, S, S(O), SO₂ or NH and one or two CH moieties unreplaced or replaced with N and also means cycloalkane having one or two or three CH₂ moieties unreplaced or replaced with independently selected O, S, S(O), SO₂ or NH and one or two CH moieties replaced with N.

The term “heterocycloalkyl,” as used herein, means cycloalkyl having one or two or three CH₂ moieties replaced with independently selected O, S, S(O), SO₂ or NH and one or two CH moieties unreplaced or replaced with N and also means cycloalkyl having one or two or three CH₂ moieties unreplaced or replaced with independently selected O, S, S(O), SO₂ or NH and one or two CH moieties replaced with N.

The term “heterocycloalkene,” as used herein, means cycloalkene having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected O, S, S(O), SO₂ or NH and one or two CH moieties replaced with N.

The term “heterocycloalkenyl,” as used herein, means cycloalkenyl having one or two or three CH₂ moieties replaced with independently selected O, S, S(O), SO₂ or NH and one or two CH moieties unreplaced or replaced with N and also means cycloalkenyl having one or two or three CH₂ moieties unreplaced or replaced with independently selected O, S, S(O), SO₂ or NH and one or two CH moieties replaced with N.

The term “alkenyl,” as used herein, means C₂-alkenyl, C₃-alkenyl, C₄-alkenyl, C₅-alkenyl and C₆-alkenyl.

The term “alkyl,” as used herein, means C₁-alkyl, C₂-alkyl, C₃-alkyl, C₄-alkyl, C₅-alkyl and C₆-alkyl.

The term “alkynyl,” as used herein, means C₂-alkynyl, C₃-alkynyl, C₄-alkynyl, C₅-alkynyl and C₆-alkynyl.

The term “C₂-alkenyl,” as used herein, means ethenyl (vinyl).

The term “C₃-alkenyl,” as used herein, means 1-propen-1-yl, 1-propen-2-yl (isopropenyl) and 1-propen-3-yl (allyl).

The term “C₄-alkenyl,” as used herein, means 1-buten-1-yl, 1-buten-2-yl, 1,3-butadien-1-yl, 1,3-butadien-2-yl, 2-buten-1-yl, 2-buten-2-yl, 3-buten-1-yl, 3-buten-2-yl, 2-methyl-1-propen-1-yl and 2-methyl-2-propen-1-yl.

The term “C₅-alkenyl,” as used herein, means 2-methylene-3-buten-1-yl, 2-methylenebut-1-yl, 2-methyl-1-buten-1-yl, 2-methyl-1,3-butadien-1-yl, 2-methyl-2-buten-1-yl, 2-methyl-3-buten-1-yl, 2-methyl-3-buten-2-yl, 3-methyl-1-buten-1-yl, 3-methyl-1-buten-2-yl, 3-methyl-1,3-butadien-1-yl, 3-methyl-1,3-butadien-2-yl, 3-methyl-2-buten-1-yl, 3-methyl-2-buten-2-yl, 3-methyl-3-buten-1-yl, 3-methyl-3-buten-2-yl, 1-penten-1-yl, 1-penten-2-yl, 1-penten-3-yl, 1,3-pentadien-1-yl, 1,3-penta-dien-2-yl, 1,3-pentadien-3-yl, 1,4-pentadien-1-yl, 1,4-pentadien-2-yl, 1,4-pentadien-3-yl, 2-penten-1-yl, 2-penten-2-yl, 2-penten-3-yl, 2,4-pentadien-1-yl, 2,4-pentadien-2-yl, 3-penten-1-yl, 3-penten-2-yl, 4-penten-1-yl and 4-penten-2-yl.

The term “C₆-alkenyl,” as used herein, means 2,2-dimethyl-3-buten-1-yl, 2,3-dimethyl-1-buten-1-yl, 2,3-dimethyl-1,3-butadien-1-yl, 2,3-dimethyl-2-buten-1-yl, 2,3-dimethyl-3-buten-1-yl, 2,3-dimethyl-3-buten-2-yl, 3,3-dimethyl-1-buten-1-yl, 3,3-dimethyl-1-buten -2-yl, 2-ethenyl-1,3-butadien-1-yl, 2-ethenyl-2-buten-1-yl, 2-ethyl-1-buten-1yl, 2-ethyl-1,3-butadien-1-yl, 2-ethyl-2-buten-1-yl, 2-ethyl-3-buten-1-yl, 1-hexen-1-1-hexen-2-yl, 1-hexen-3-yl, 1,3-hexadien-1-yl, 1,3-hexadien-2-yl, 1,3-hexadien-3-yl, 1,3,5-hexatrien-1-yl, 1,3,5-hexatrien-2-yl, 1,3,5-hexatrien-3-yl, 1,4-hexadien-1-yl, 1,4-hexadien-2-yl, 1,4-hexadien-3-yl, 1,5-hexadien-1-yl, 1,5-hexadien-2-yl, 1,5-hexadien-3-yl, 2-hexen-1-yl, 2-hexen-2-yl, 2-hexen-3-yl, 2,4-hexadien-1-yl, 2,4-hexadien-2-yl, 2,4-hexadien-3-yl, 2,5-hexadien-1-yl, 2,5-hexadien-2-yl, 2,5-hexadien-3-yl, 3-hexen-1-yl, 3-hexen-2-yl, 3-yl, 3,5-hexadien-1-yl, 3,5-hexadien-2-yl, 3,5-hexadien-3-yl, 4-hexen-1-yl, 4-hexen-2-yl, 4-hexen-3-yl, 5-hexen-1-yl, 5-hexen-2-yl, 5-hexen-3-yl, 2-methylene-3-methyl-3-buten-1-yl, 2-methylene-3-methylbut-1-yl, 2-methylene-3-penten-1-yl, 2-methylene-4-penten-1-yl, 2-methylenepent-1-yl, 2-methylenepent-3-yl, 3-methylene-1-penten-1-yl, 3-methylene-1-penten-2-yl, 3-methylenepent-1-yl, 3-methylene-1,4-pentadien-1-yl, 3-methylene-1,4-pentadien-2-yl, 3-methylene-pent-2-yl, 2-methyl-1-penten-1-yl, 2-methyl-1-penten-3-yl, 2-methyl-1,3-pentadien-1-yl, 2-methyl-1,3-pentadien-3-yl, 2-methyl-1,4-pentadien-1-yl, 2-methyl-1,4-pentadien-3-yl, 2-methyl-2-penten-1-yl, 2-methyl-2-penten-3-yl, 2-methyl-2,4-pentadien-1-yl, 2-methyl-2,4-pentadien-3-yl, 2-methyl-3-penten-1-yl, 2-methyl-3-penten-2-yl, 2-methyl-3-penten-3-yl, 2-methyl-4-penten-1-yl, 2-methyl-4-penten-2-yl, 2-methyl-4-penten-3-yl, 3-methyl-1-penten-1-yl, 3-methyl-1-penten-2-yl, 3-methyl-1,3-pentadien-1-yl, 3-methyl-1,3-pentadien-2-yl, 3-methyl-1,4-pentadien-1-yl, 3-methyl-1,4-pentadien-2-yl, 3-methyl-2-penten-1-yl, 3-methyl-2-penten-2-yl, 3-methyl-2,4-pentadien-1-yl, 3-methyl-3-penten-1yl, 3-methyl-3-penten-2-yl, 3-methyl-4-penten-1-yl, 3-methyl-4-penten-2-yl, 3-methyl-4-penten-3-yl, 4-methyl-1-penten-1-yl, 4-methyl-1-penten-2-yl, 4-methyl-i -penten-3-yl, 4-methyl-1,4-pentadien-1-yl, 4-methyl-1,3-pentadien-2-yl, 4-methyl-1,3-pentadien-3-yl, 4-methyl-1,4-pentadien-1-yl, 4-methyl-1,4-pentadien-2-yl, 4-methyl-1,4-pentadien-3-yl, 4-methylene-2-penten-3-yl, 4-methyl-2-penten-1-yl, 4-methyl-2-penten-2-yl, 4-methyl-2-penten-3-yl, 4-methyl-2,4-pentadien-1-yl, 4-methyl-2,4-pentadien-2-yl, 4-methyl-3-penten-1-yl, 4-methyl-3-penten-2-yl, 4-methyl-3-penten-3-yl, 4-methyl-4-penten-1-yl and 4-methyl-4-penten-2yl.

The term “C₁-alkyl,” as used herein, means methyl.

The term “C₂-alkyl,” as used herein, means ethyl.

The term “C₃-alkyl,” as used herein, means prop-1-yl and prop-2-yl (isopropyl).

The term “C₄-alkyl,” as used herein, means but-1-yl, but-2-yl, 2-methylprop-1-yl and 2-methylprop-2-yl (tert-butyl).

The term “C₅-alkyl,” as used herein, means 2,2-dimethylprop-1-yl (neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein, means 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl and 4-methylpent-2-yl.

The term “C₂-alkynyl,” as used herein, means ethynyl (acetylenyl).

The term “C₃-alkynyl,” as used herein, means 1-propyn-1-yl and 2-propyn-1-yl (propargyl).

The term “C₄-alkynyl,” as used herein, means 1-butyn-1-yl, 1,3-butadiyn-1-yl, 2-butyn-1-yl, 3-butyn-1-yl and 3-butyn-2-yl.

The term “C₅-alkynyl,” as used herein, means 2-methyl-3-butyn-1-yl, 2-methyl-3-butyn-2-yl, 3-methyl-1-butyn-1-yl, 1,3-pentadiyn-1-yl, 1,4-pentadiyn-1-yl, 1,4-pentadiyn-3-yl, 2,4-pentadiyn-1-yl, 1-pentyn-1-yl, 1-pentyn-3-yl, 2-pentyn-1-yl, 3-pentyn-1-yl, 3-pentyn-2-yl, 4-pentyn-1-yl and 4-pentyn-2-yl.

The term “C₆-alkynyl,” as used herein, means 2,2-dimethyl-3-butyn-1-yl, 3,3-dimethyl-1-butyn-1-yl, 2-ethyl-3-butyn-1-yl, 2-ethynyl-3-butyn-1-yl, 1-hexyn-1-yl, 1-hexyn-3-yl, 1,3-hexadiyn-1-yl, 1,3,5-hexatriyn-1-yl, 1,4-hexadiyn-1-yl, 1,4-hexadiyn-3-yl, 1,5-hexadiyn-1-yl, 1,5-hexadiyn-3-yl, 2-hexyn-1-yl, 2,5-hexadiyn-1-yl, 3-hexyn-1-yl, 3-hexyn-2-yl, 3,5-hexadiyn-2-yl, 4-hexyn-1-yl, 4-hexyn-2-yl, 4-hexyn-3-yl, 5-hexyn-1-yl, 5-hexyn-2-yl, 5-hexyn-3-yl, 2-methyl-3-pentyn-1-yl, 2-methyl-3-pentyn-2-yl, 2-methyl-4-pentyn-1-yl, 2-methyl-4-pentyn-2-yl, 2-methyl-4-pentyn-3-yl, 3-methyl-1-pentyn-1-yl, 3-methyl-4-pentyn-1-yl, 3-methyl-4-pentyn-2-yl, 3-methyl-1,4-pentadiyn-1-yl, 3-methyl-1,4-pentadiyn-3-yl, 3-methyl-4-pentyn-1-yl, 3-methyl-4-pentyn-3-yl, 4-methyl-1-pentyn-1-yl and 4-methyl-2-pentyn-1-yl.

The term “C₄-cycloalkane,” as used herein, means cyclobutane.

The term “C₅-cycloalkane,” as used herein, means cyclopentane.

The term “C₆-cycloalkane,” as used herein, means cyclohexane.

The term “C₄-cycloalkene,” as used herein, means cyclobutene and 1,3-cyclobutadiene.

The term “C₅-cycloalkene,” as used herein, means cyclopentene and 1,3-cyclopentadiene.

The term “C₆-cycloalkene,” as used herein, means cyclohexene, 1,3-cyclohexadiene and 1,4-cyclohexadiene.

The term “C₃-cycloalkenyl,” as used herein, means cycloprop-1-en-1-yl and cycloprop-2-en-1-yl.

The term “C₄-cycloalkenyl,” as used herein, means cyclobut-1-en-1-yl and cyclobut-2-en-1-yl.

The term “C₅-cycloalkenyl,” as used herein, means cyclopent-1-en-1-yl, cyclopent-2-en-1-yl, cyclopent-3-en-1-yl and cyclopenta-1,3-dien-1-yl.

The term “C₆-cycloalkenyl,” as used herein, means cyclohex-1-en-1-yl, cyclohex-2-en-1-yl, cyclohex-3-en-1-yl, cyclohexa-1,3-dien-1-yl, cyclohexa-1,4-dien-1-yl, cyclohexa-1,5-dien-1-yl, cyclohexa-2,4-dien-1-yl and cyclohexa-2,5-dien-1-yl.

The term “C₃-cycloalkyl,” as used herein, means cycloprop-1-yl.

The term “C₄-cycloalkyl,” as used herein, means cyclobut-1-yl.

The term “C₅-cycloalkyl,” as used herein, means cyclopent-1-yl.

The term “C₆-cycloalkyl,” as used herein, means cyclohex-1-yl.

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds of this invention may also exist as a mixture of “Z” and “E” isomers.

Compounds of this invention may also exist as tautomers or equilibrium mixtures thereof wherein a proton of a compound shifts from one atom to another. Examples of tautomers include, but are not limited to, keto-enol, phenol-keto, oxime-nitroso, nitro-aci, imine-enamine and the like.

Compounds of this invention containing NH, C(O)OH, OH or SH moieties may have attached thereto prodrug-forming moieties. The prodrug-forming moieties are removed by metabolic processes and release the compounds having the freed NH, C(O)OH, OH or SH in vivo. Prodrugs are useful for adjusting such pharmacokinetic properties of the compounds as solubility and/or hydrophobicity, absorption in the gastrointestinal tract, bioavailability, tissue penetration, and rate of clearance.

Metabolites of compounds having formula (I), formula (II) or formula (III), produced by in vitro or in vivo metabolic processes, may also have utility for treating diseases associated with overexpression of HSP90.

Certain precursor compounds which may be metabolized in vitro or in vivo to form compounds having formula (I), formula (II) or formula (III) may also have utility for treating diseases associated with overexpression of HSP90.

Compounds having formula (I), formula (II) or formula (III) may exist as acid addition salts, basic addition salts or zwitterions. Salts of compounds having formula (I), formula (II) or formula (III) are prepared during their isolation or following their purification. Acid addition salts are those derived from the reaction of a compound having formula (I), formula (II) or formula (III) with acid. Accordingly, salts including the acetate, adipate, alginate, bicarbonate, citrate, aspartate, benzoate, benzenesulfonate (besylate), bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate and undecanoate salts of the compounds having formula (I), formula (II) or formula (III) are meant to be embraced by this invention. Basic addition salts of compounds are those derived from the reaction of the compounds having formula (I), formula (II) or formula (III) with the bicarbonate, carbonate, hydroxide or phosphate of cations such as lithium, sodium, potassium, calcium and magnesium.

Compounds having formula (I), formula (II) or formula (III) may be administered, for example, bucally, ophthalmically, orally, osmotically, parenterally (intramuscularly, intraperintoneally intrasternally, intravenously, subcutaneously), rectally, topically, transdermally, vaginally and intraarterially as well as by intraarticular injection, infusion, and placement in the body, such as, for example, the vasculature.

Therapeutically effective amounts of a compound having formula (I), formula (II) or formula (III) depend on recipient of treatment, disease treated and severity thereof, composition comprising it, time of administration, route of administration, duration of treatment, potency, rate of clearance and whether or not another drug is co-administered. The amount of a compound having formula (I) used to make a composition to be administered daily to a patient in a single dose or in divided doses is from about 0.03 to about 200 mg/kg body weight. Single dose compositions contain these amounts or a combination of submultiples thereof.

Compounds having formula (I), formula (II) or formula (III) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising a compound having formula (I), formula (II) or formula (III) to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl flimarate, sucrose, surfactants, talc, tragacanth, tetrahydrofurfuryl alcohol, triglycerides, water, mixtures thereof and the like. Excipients for preparation of compositions comprising a compound having formula (I), formula (II) or formula (III) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I), formula (II) or formula (III) to be administered osmotically include, but are not limited to, chlorofluorohydrocarbons, ethanol, water, mixtures thereof and the like. Excipients for preparation of compositions comprising a compound having formula (I), formula (II) or formula (III) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I), formula (II) or formula (III) to be administered rectally or vaginally include, but are not limited to, cocoa butter, polyethylene glycol, wax, mixtures thereof and the like.

Compounds having formula (I), formula (II) or formula (III) are also expected to be useful as chemotherapeutic agents in combination with actinomycins, alkylating agents, anthracyclines, antifolates, antiestrogen agents, anti-metabolites, anti-androgens, antimicrotubule agents, aromatase inhibitors, bleomycins, Ca²⁺ adenosine triphosphate (ATP)ase inhibitors, cytosine analogs, deltoids/retinoids, dihydrofolate reductase inhibitors, deoxyribonucleic acid (DNA) topoisomerase inhibitors, dopaminergic neurotoxins, glucocorticoids, histone deacetylase inhibitors, hormonal therapies, immunotherapeutic agents, inosine monophosphate (IMP) dehydrogenase inhibitors, isoprenylation inhibitors, luteinizing hormone-releasing hormone agonists, mammalian target of rapamycin (mtor) inhibitors, multi-drug resistance (MDR) inhibitors, mitomycins, photodyamic therapies, proteasome inhibitors, platinum containing compounds, radiation, receptor tyrosine kinase inhibitors, ribonuclotide reductase inhibitors, thrombospondin mimetics, uracil analogs, vinca alkaloids, and vitamin D3 analogs such as, but not limited to, y-radiation or an additional chemotherapeutic agent or additional chemotherapeutic agents such as N-Ac-Sar-Gly-Val-D-alloIle-Thr-Nva-Ile-Arg-Pro-NHCH₂CH₃ or a salt thereof, actinomycin D, AG13736, 17-allylamino-17-demethoxygeldanamycin, 9-aminocamptothecin, N-(4-(3-amino-1H-indazol-4-yl)phenyl)-N′-(2-fluoro-5-methylphenyl)urea or a salt thereof, N-(4-(4-aminothieno[2,3-d]pyrimidin-5-yl)phenyl)-N′-(2-fluoro-5-(trifluoromethyl)phenyl)urea or a salt thereof, anastozole, AP-23573, asparaginase, azacitidine, bevacizumab, bicalutamide, bleomycin a2, bleomycin b2, bortezamib, busulfan, campathecins, carboplatin, carmustine (BCNU), CB1093, cetuximab, CHOP (C: Cytoxan(® (cyclophosphamide); H: Adriamycin® (hydroxydoxorubicin); O: Vincristine (Oncovin®); P: prednisone), chlorambucil, CHIR258, cisplatin, CNF-101, CNF-1001, CNF-2024, CP547632, crisnatol, cytarabine, cyclophosphamide, cytosine arabinoside, daunorubicin, dacarbazine, dactinomycin, dasatinib, daunorubicin, deferoxamine, demethoxyhypocrellin A, depsipeptide, dexamethasone, 17-dimethylaminoethylamino-17-demethoxygeldanamycin, docetaxel, doxifluridine, doxorubicin, EB1089, epothilone D, epirubicin, 5-ethynyl-1-β-D-ribofuranosylimidazole-4-carboxamide (EICAR), erlotinib, etoposide, everolimus, 5-fluorouracil (5-FU), floxuridine, fludarabine, flutamide, gefitinib, geldanamycin, gemcitabine, goserelin, N-(2-(4-hydroxyanilino)-3-pyridinyl)-4-methoxybenzenesulfonamide or a salt thereof, hydroxyurea, idarubicin, ifosfamide, imatinab, interferon-α, interferon-γ, IPI-504, irinotecan, KH 1060, lapatanib, LAQ824, leuprolide acetate, letrozole, lomustine (CCNU), lovastatin, megestrol, melphalan, mercaptopurine, methotrexate, 1-methyl-4-phyenylpyridinium, MG132, mitomycin, mitoxantrone, MLN-518, MS-275, mycophenolic acid, mitomycin C, nitrosoureas, oxaliplatin, paclitaxel, PD98059, peplomycin, photosensitizer Pc4, phtalocyanine, pirarubicin, plicamycin, prednisone, procarbizine, PTK787, PU24FCl, PU3, radicicol, raloxifene, rapamycin, ratitrexed, retinoids such as pheuretinide, ribavirin, rituximab (Rituxin®), sorafenib, staurosporine, steroids such as dexamethasone and prednisone, suberoylanilide hydroxamic acid, sunitinib, tamoxifen, taxol, temozolamide, temsirolimus, teniposide, thapsigargin, thioguanine, thrombospondin-1, tiazofurin, topotecan, trapoxin, trastuzumab, treosulfan, trichostatin A, trimetrexate, trofosfamide, tumor necrosis factor, valproic acid, VER49009, verapamil, vertoporfin, vinblastine, vincristine, vindesine, vinorelbine vitamin D3, VX-680, zactima, ZK-EPO, zorubicin or combinations thereof.

Binding affinity of compounds having formula (I), formula (II) or formula (III) to HSP90 is indicia of their inhibititory activity of this protein. To determine the binding affinity of compounds having formula (I) to HSP90, an HSP90-FRET assay was used.

Assay buffer used was (62.5 mM Tris (pH 7.5),187.5 mM NaCl, 0.0625% triton X-100, 1.25 mM DTT, 1.25 mM EDTA) and cocktail (from mixing Eu-Ab, SA-APC, GM-biotin and HSP90) with the assay buffer according to following formula: for each reaction (40 μL/well), needed 0.04 μL Eu-Ab, 0.33 μL SA-APC, 0.016 μL GM-biotin, 0.010 μL HSP90 (194 μM) and 39.6 μL of buffer. Thus, the final concentration of each component in the assay reaction was 50 mM Tris (pH 7.5), 150 mM NaCl, 1 mM EDTA, 1 mM DTT, 0.05% triton X-100, 0.04 μM HSP90, 0.08 μM GM-biotin, 0.08 μM SA-APC and 0.001 μM Eu-Ab.

Representative compounds were dissolved in DMSO to 10 mM. DMSO was then used to prepare a series dilution with a 1:3 ratio in each step, wherein, upon completion, the highest concentration was 2500 μM. Water was then used to make another 1:10 dilution for all of the previous dilutions, so that the highest concentration was 250 μM. 10 μL of each water dilution was transferred into each well of a 96 well plate. 40 μL of assay cocktail solution was added to each well, and the solutions were shaken for 4 hours at room temperature. Emission at 650 nm and 615 nm were measured using a Perkin Elmer Envision plate reader. Bacterially expressed N-ter human HSP90 (9-236aa) was used in this assay.

Ki values, calculated using Microsoft Excel, were 0.004 μM, 0.02 μM, 0.02 μM,
0.029 μM, 0.03 μM, 0.03 μM, 0.03 μM, 0.04 μM, 0.04 μM,
0.04 μM, 0.04 μM, 0.05 μM, 0.05 μM, 0.05 μM, 0.05 μM,
0.05 μM, 0.05 μM, 0.06 μM, 0.06 μM, 0.06 μM, 0.06 μM,
0.06 μM, 0.07 μM, 0.08 μM, 0.09 μM, 0.10 μM, 0.10 μM,
0.10 μM, 0.10 μM, 0.10 μM, 01.0 μM, 0.11 μM, 0.11 μM,
0.12 μM, 0.13 μM, 0.14 μM, 0.15 μM, 0.16 μM, 0.16 μM,
0.16 μM, 0.17 μM, 0.19 μM, 0.21 μM, 0.21 μM, 0.22 μM,
0.23 μM, 0.25 μM, 0.25 μM, 0.28 μM, 0.28 μM, 0.28 μM,
0.28 μM, 0.39 μM, 0.40 μM, 0.40 μM, 0.42 μM, 0.45 μM,
0.45 μM, 0.48 μM, 0.49 μM, 0.49 μM, 0.50 μM, 0.51 μM,
0.56 μM, 0.56 μM, 0.58 μM, 0.59 μM, 0.61 μM, 0.77 μM,
0.79 μM, 0.86 μM, 0.93 μM, 1.03 μM, 1.08 μM, 1.16 μM,
1.25 μM, 1.33 μM, 1.34 μM, 1.38 μM, 1.46 μM, 1.52 ,μM,
1.58 μM, 1.73 μM, 1.86 μM, 1.90 μM, 1.96 μM, 2.10 μM,
2.14 μM, 2.19 μM, 2.46 μM, 2.64 μM, 3.07 μM, 3.39 μM,
3.74 μM, 4.38 μM, 4.89 μM, 5.37 μM, 5.70 μM, 6.14 μM,
6.30 μM, 6.59 μM, 6.75 μM, 6.90 μM, 7.62 μM, 8.26 μM,
8.45 μM, 9.40 μM, 9.89 μM, 10.17 μM, 10.50 μM, 13.36 μM,
13.85 μM, 18.79 μM, 21.81 μM and 24.86 μM

To determine the binding affinity of compounds having formula (I), formula (II) or formula (III) to HSP90, an malachite green assay from Analytical Biochemistry 327 (2004) 176-183 was also used.

Assay buffer used was 100 mM Tris-HCl, pH 7.4, 20 mM KCl and 6 mM MgCl₂.

Serial dilutions of test compounds in 96 well plates were made in 100% DMSO. ATP, sodium salt was dissolved in assay buffer to provide a stock concentration of 1.923 mM and stored at room temperature on day of the experiment. An aliquot of the ATP solution (13 μL) was added to each well to give a final assay concentration of 1 mM. 2 μL of diluted test compounds or DMSO (control) were added to each well. Just before use, yeast HSP90 protein on ice was thawed and diluted in chilled assay buffer to a stock concentration of 0.30 mg/mL and kept on ice. Incubation was started by adding 10 μL of the diluted HSP90 to each well (except for the background wells which received 10 μL assay buffer) to provide a final assay volume of 25 μL and 3 μg HSP90/well. The plates were sealed with plastic film, shaken for 2 minutes and incubated for 3 hours at 37° C. Malachite green solutions A and B (Upstate Cell Signalling, Cat#20-105, and 20-104, respectively) were warmed to room temperature. Solution B (Tween-20, 10 μL) was added to solution A (malachite green) (1 mL) to activate the malachite green. To stop the reaction, a 80 μL of activated malachite green reagent was added to each well of the plate, and the plate was shaken again. Following the addition of 10 μL of 34% sodium citrate additive to each well, the plates were again shaken and allowed to stand at room temperature for 15 minutes. Absorbance was read at 620 nm.

Ki values, calculated using Microsoft Excel, were 0.25 μM, 0.32 μM, 0.34 μM,
0.34 μM, 0.35 μM, 0.42 μM 0.48 μM 0.48 nM 0.58 μM
0.60 μM, 0.61 μM 0.63 μM 0.64 μM 0.64 μM 0.65 μM
0.67 μM, 0.68 μM 0.69 μM 0.72 μM 0.76 μM 0.77 μM
0.79 μM, 0.81 μM 0.86 μM 0.90 μM 0.94 μM 1.03 μM
1.12 μM, 1.14 μM 1.20 μM 1.31 μM 1.36 μM 1.37 μM
1.38 μM, 1.49 μM, 1.58 μM, 1.60 μM, 1.62 μM, 1.63 μM,
1.64 μM, 1.69 μM, 1.76 μM, 1.77 μM, 1.82 μM, 1.91 μM,
1.93 μM, 2.09 μM, 2.35 μM, 2.43 μM, 2.62 μM, 2.69 μM,

02.81 μM, 2.96 μM, 3.64 μM, 4.30 μM, 4.43 μM, 4.57 μM,
4.85 μM, 4.94 μM, 5.64 μM, 5.82 μM, 6.33 μM, 6.87 μM,
7.32 μM, 7.59 μM, 7.61 μM, 7.98 μM, 8.54 μM, 8.66 μM,
8.67 μM, 8.80 μM, 8.80 μM, 8.96 μM, 9.33 μM, 10.44 μM,
11.08 μM, 12.29 μM, 12.30 μM, 12.48 μM, 12.72 μM, 13.19 μM,
14.17 μM, 14.21 μM, 15.18 μM, 15.35 μM, 17.56 μM, 17.57 μM,
18.62 μM, 20.70 μM, 21.61 μM, 22.87 μM, 22.88 μM, 24.82 μM,
24.90 μM, 25.22 μM, 25.53 μM, 26.24 μM, 28.51 μM, 32.72 μM,
33.06 μM, 49.10 μM, 58.49 μM, 67.03 μM, 69.65 μM, 75.03 μM,
81.98 μM, 85.04 μM, 90.46 μM, 122 μM, 200 μM, 200 μM,
200 μM, 200 μM and 200 μM.

These data demonstrate the utility of compounds having formula (I), formula (II) or formula (III) as binders to and inhibitors of HSP90.

Diseases which may be exacerbated by involvement with HSP90, include, but are not limited to cancer and autoimmune disorders, wherein cancer includes, but is not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, renal cell carcinoma, retinoblastoma, rhabdomyosarcoma, sarcoma, sebaceous gland carcinoma, seminoma, small cell lung carcinoma, solid tumors (carcinomas and sarcomas), small cell lung cancer squamous cell carcinoma, synovioma, sweat gland carcinoma, Waldenstrom's macroglobulinemia, testicular tumors, uterine cancer and Wilms' tumor.

It is also expected that compounds having formula (I), formula (II) or formula (III) would inhibit the growth of cells derived from a cancer or neoplasm such as breast cancer (including estrogen-receptor positive breast cancer), colorectal cancer, endometrial cancer, lung cancer (including small cell lung cancer), lymphoma (including follicular or Diffuse Large B-cell), lymphoma (including non-Hodgkin's lymphoma), neuroblastoma, ovarian cancer, prostate cancer (including hormone-insensitive prostate cancer) and testicular cancer (including germ cell testicular cancer).

It is also expected that compounds having formula (I), formula (II) or formula (III) would inhibit the growth of cells derived from a pediatric cancer or neoplasm such as embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous syatem, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer.

Involvement of HSP90 in acute lymphocytic leukemia is demonstrated in Leukemia, 2001, 15, 1537-1543.

Involvement of HSP90 in breast cancer is demonstrated in Brit. J. Cancer, 1996, 74, 717-721; Clin Cancer Res 2003, 9, 4961-4971; Cancer Res., 2000, 60, 2232-2238; Int. J. Cancer, 1992, 50, 409-415; and Oncogene, 1991, 6, 1125-1132.

Involvement of HSP90 in cervical cancer is demonstrated in Cancer Res. 2003, 63, 8984-8995.

Involvement of HSP90 in chronic myelogenous leukemia is demonstrated in Leukemia, 2001, 15, 1537-1543.

Involvement of HSP90 in colon cancer is demonstrated in J. Natl. Cancer Inst 1999; 91, 1940-1949.

Involvement of HSP90 in lung cancer is demonstrated in Ann. Thorac. Surg., 2001, 72,271-379.

Involvement of HSP90 in melanoma is demonstrated in Cancer Chemother. Pharmacol, 2005, 56, 115-125.

Involvement of HSP90 in ovarian cancer is demonstrated in Cancer Res., 2005, 11, 7023-7032.

Involvement of HSP90 inhibitors in pancreatic cancer is demonstrated in Cancer Chemother. Pharmacol, 2005, 56, 115-125.

Involvement of HSP90 in prostate cancer is demonstrated in Clin. Cancer Res. 2004, 10, 8077-8084; Am. J. Pathol., 2000, 156, 857-864; and Clin. Cancer Res., 2002, 8, 986-993.

Involvement of HSP90 in renal carcinoma is demonstrated in J. Biol. Chem. 2002, 277, 29936-29944.

Involvement of HSP90 in squamous cell carcinoma is demonstrated in Clin. Cancer Res. 2005, 11, 3889-3896 and Cancer, 1999, 85, 1649-1657.

Compounds having formula (I), formula (II) or formula (III) may be made by synthetic chemical processes, examples of which are shown hereinbelow. It is meant to be understood that the order of the steps in the processes may be varied, that reagents, solvents and reaction conditions may be substituted for those specifically mentioned, and that vulnerable moieties may be protected and deprotected, as necessary.

Protecting groups for C(O)OH moieties include, but are not limited to, acetoxymethyl, allyl, benzoylmethyl, benzyl, benzyloxymethyl, tert-butyl, tert-butyldiphenylsilyl, diphenylmethyl, cyclobutyl, cyclohexyl, cyclopentyl, cyclopropyl, diphenylmethylsilyl, ethyl, para-methoxybenzyl, methoxymethyl, methoxyethoxymethyl, methyl, methylthiomethyl, naphthyl, para-nitrobenzyl, phenyl, n-propyl, 2,2,2-trichloroethyl, triethylsilyl, 2-(trimethylsilyl)ethyl, 2-(trimethylsilyl)ethoxymethyl, triphenylmethyl and the like.

Protecting groups for C(O) and C(O)H moieties include, but are not limited to, 1,3-dioxylketal, diethylketal, dimethylketal, 1,3-dithianylketal, O-methyloxime, O-phenyloxime and the like.

Protecting groups for NH moieties include, but are not limited to, acetyl, alanyl, benzoyl, benzyl (phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, formyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

A preferred protecting group for phenolic OH moieties is methyl.

The following abbreviations have the meanings indicated. ADDP means 1,1′-(azodicarbonyl)dipiperidine; AD-mix-β means a mixture of (DHQD)₂PHAL, K₃Fe(CN)₆, K₂CO₃ and K₂SO₄); AIBN means 2,2′-azobis(2-methylpropionitrile); 9-BBN means 9-borabicyclo[3.3.1]nonane; (DHQD)₂PHAL means hydroquinidine 1,4-phthalazinediyl diethyl ether; DBU means 1,8-diazabicyclo[5.4.0]undec-7-ene; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppa means diphenylphosphoryl azide; dppb means 1,4-bis(diphenylphosphino)butane; dppe means 1,2-bis(diphenylphosphino)ethane; dppf means 1,1′-bis(diphenylphosphino)ferrocene; dppm means 1,1-bis(diphenylphosphino)methane; EDAC means 1-(3-dimethylaminopropyl)-3-ethylcarbodiimide; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; LDA means lithium diisopropylamide; LHMDS means lithium bis(hexamethyldisilylamide); MP-BH₃ means macroporus triethylammonium methylpolystyrene cyanoborohydride; LAH means lithium aluminum hydride; NCS means N-chlorosuccinimide; PyBOP means benzotriazol-1-yloxytripyrrolidinophosphonium hexafluorophosphate; TDA-1 means tris(2-(2-methoxyethoxy)ethyl)amine; TEA means triethylamine; TFA means trifluoroacetic acid; THF means tetrahydrofuran; NCS means N-chlorosuccinimide; NMM means N-methylmorpholine; NMP means N-methylpyrrolidine;

PPh₃ means triphenylphosphine.

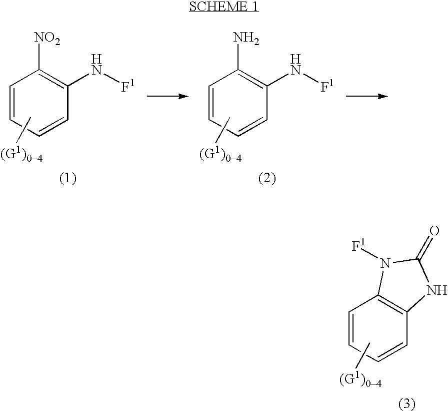

As shown in SCHEME 1, compounds having formula (1), wherein G¹ represents independently selected R¹², OR¹², SR¹², S(O)R¹², SO₂R¹², NH₂, NHR¹², N(R¹²)₂, N(CH₃)(R¹²)₂, C(O)R¹², C(O)NH₂, C(O)NHR¹², C(O)N(R¹²)₂, NHC(O)R¹², NR¹²C(O)R¹², NHSO₂R¹², NR¹²SO₂R¹², NHC(O)OR¹², NR¹²C(O)OR¹², SO₂NH₂, SO₂NHR¹², SO₂N(R¹²)₂, NHC(O)NH₂, NHC(O)NHR¹², NHC(O)N(R¹²)₂, NR¹²C(O)N(R¹²)₂, OH, (O), C(O)H, C(O)OH, NO₂, CN, CF₃, CF₂CF₃, F, Cl, Br or I, may be converted to compounds having formula (2) by reacting the former, hydrogen or a hydrogen source and a reduction catalyst. An example of a hydrogen source is cyclohexene. Examples of reduction catalysts include, but are not limited to, platinum, palladium, platinum on carbon, palladium on carbon and raney nickel. The reaction is typically conducted in a solvent such as methanol, ethanol, THF, ethyl acetate or mixtures thereof at temperatures between about 75° C. and 175° C.

Compounds having formula (2) may be converted to compounds having formula (3) by reacting the former and a cyclizing agent, with or without a base. Examples of cyclizing agents incluse phosgene and diethyl carbonate. Examples of bases include TEA, DIEA, and potassium carbonate.

Reaction conditions depend on reagent used. For example, when phosgene is used, the reaction is typically conducted in a solvent such as chloroform, dichloromethane benzene, toluene or mixtures thereof at temperatures between about 0° C. and 50° C. When diethyl carbonate is used, the reaction is typically conducted with potassium carbonate and without solvent at temperatures between about 750° C. and 100° C.

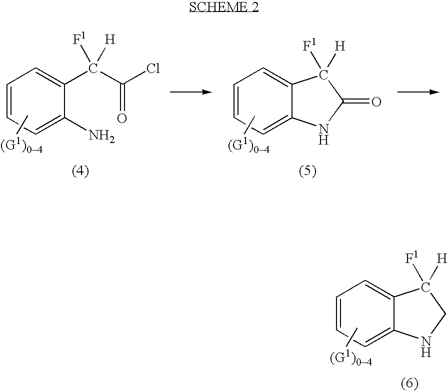

As shown in SCHEME 2, compounds having formula (4) may be converted to compounds having formula (5) by intramolecular cyclization, with or without a base. Examples of bases include TEA, DIEA and potassium carbonate. The reaction is typically conducted in a solvent such as THF, DMF, DMSO, ethyl acetate or mixtures thereof at temperatures between about 25° C. and 75° C.

Compounds having formula (4) may be converted to compounds having formula (5) by reacting the former and a reducing agent. Examples of reducing agents include BH₃-THF, DIBAL and LAH. The reaction is typically conducted in a solvent such as diethyl ether, THF, hexanes, or dichloromethane at temperatures between about 25° C. and 75° C.

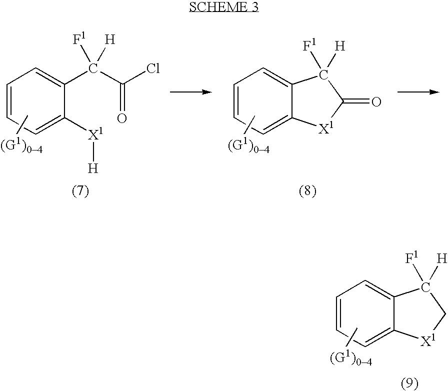

As shown in SCHEME 3, compounds having formula (7) may be converted to compounds having formula (8) using the same reagents and under the same reaction conditions as shown for the conversion of compounds having formula (4) to compounds having formula (5) in SCHEME 2. Compounds having formula (8) may be converted to compounds having formula (9) using the same reagents and under the same reaction conditions as shown for the conversion of compounds having formula (5) to compounds having formula (6) in SCHEME 2.

The following examples are presented to provide what is believed to be the most useful and readily understood description of procedures and conceptual aspects of this invention.

### EXAMPLE 1A

A suspension of 1-fluoro-2-nitro-4-trifluoromethylbenzene (0.3 mL), 5-chloro-2,4-dimethoxyphenylamine (0.4 g) and potassium fluoride (0.2 g) in DMA (1.5 mL) at 150° C. was stirred under microwaves for 10 minutes, diluted with ethyl acetate, washed with water and concentrated. The concentrate was recrystallized from ethanol.

### EXAMPLE 1B

A suspension of EXAMPLE 1A (0.085 g), 10% Pd/C (25 mg) and cyclohexene (0.2 mL) in ethanol (8 mL) at 150° C. was stirred under microwaves for 10 minutes, filtered and concentrated. The concentrate was dissolved in dichloromethane and the solution was treated with 20% phosgene in toluene (0.5 mL), stirred at ambient temperature for 16 hours and concentrated. The concentrate was purified by preparative reverse phase HPLC (Zorbax SB, C-18, 20-100% acetonitrile/water/O. 1% TFA).

### EXAMPLE 1C

To a suspension of EXAMPLE 1B (0.02 g) in dichloromethane (1 mL), a solution of 1M boron tribromide in hexane (0.2 mL) was added. The mixture was stirred at ambient temperature for 18 hours and concentrated. The concentrate was purified by preparative reverse phase HPLC (Zorbax SB, C-18, 20-100% acetonitrile/water/0.1% TFA) providing EXAMPLE 1C. ¹H NMR (500 MHz, DMSO-d₆) δ 11.30 (brs, 1H), 10.42 (brs, 1H), 10.01 (brs, 1H), 7.29 (m, 3H), 6.76 (d, 1H), 6.71 (s, 1H).

### EXAMPLE 2A

This compound was made by substituting 2,4-dimethoxy-phenylamine for 5-chloro-2,4-dimethoxy-phenylamine in EXAMPLE 1A.

### EXAMPLE 2B

This compound was made by substituting EXAMPLE 2A for EXAMPLE 1A in EXAMPLE 1B.

### EXAMPLE 2C

This compound was made by substituting EXAMPLE 2B for EXAMPLE 1B in EXAMPLE 1C. ¹H NMR (400 MHz, DMSO-d₆) δ 11.22 (s, 1H), 9.86 (s, 1H), 9.60 (brs, 1H), 7.29 (d, 1H), 7.25 (s, 1H), 7.04 (d, 1H), 6.71 (d, 1H), 6.49 (d, 1H), 6.35 (dd, 1H).

### EXAMPLE 3A

This compound was made by substituting 1-fluoro-2-nitro-benzene for 1-fluoro-2-nitro-4-trifluoromethyl-benzene in EXAMPLE 1A.

### EXAMPLE 3B

This compound was made by substituting EXAMPLE 3A for EXAMPLE 1A in EXAMPLE 1B.

### EXAMPLE 3C

This compound was made by substituting EXAMPLE 3B for EXAMPLE 1B in EXAMPLE 1C. ¹H NMR (400 MHz, DMSO-d₆) δ 10.88 (s, 1H), 10.30 (s, 1H), 9.87 (s, 1H), 7.22 (d, 1H), 7.01-6.89 (m, 3H), 6.70 (s, 1H), 6.58 (d, 1H).

### EXAMPLE 4A

This compound was made by substituting 2,4-dimethoxy-phenylamine for 5-chloro-2,4-dimethoxy-phenylamine and 1-fluoro-2-nitro-benzene for 1-fluoro-2-nitro-4-trifluoromethyl-benzene in EXAMPLE IA.

### EXAMPLE 4B

A suspension of EXAMPLE 4A (0.085 g) and 10% Pd/C (0.05 g) in methanol (10 mL) was stirred for 16 hours, filtered and concentrated. The concentrate was dissolved in dichloromethane, treated with 20% phosgene in toluene (0.25 mL), stirred for 16 hours and concentrated. The concentrate was purified by preparative reverse phase HPLC (Zorbax SB, C-18, 20-100% acetonitrile/water/O. 1% TFA).

### EXAMPLE 4C

This compound was made by substituting EXAMPLE 4B for EXAMPLE 1B in EXAMPLE 1C. ¹H NMR (500 MHz, DMSO-d₆) δ 10.83 (s, 1H), 9.58 (s, 1H), 9.54 (s, 1H), 7.02-6.95 (m, 3H), 6.91 (td, 1H), 6.53 (d, 1H), 6.47 (d, 1H), 6.33 (dd, 1H).

### EXAMPLE 5A

Benzyltrimethylammonium tribromide in 1:1 dichloromethane/methanol (20 mL) was added to a solution of EXAMPLE 2B in 1:1 dichloromethane/methanol (100 mL) over 1.5 hours. The solution was stirred for 1 hour and filtered. The filtrant was suspended in 1:1 dichloromethane/methanol (20 mL), filtered and washed with 1:1 dichloromethane/methanol (10 mL). The filtrate was concentrated, and the concentrate was suspended in 1:1 dichloromethane/methanol (20 mL) and filtered.

### EXAMPLE 5B

This compound was made by substituting EXAMPLE 5A for EXAMPLE 1B in EXAMPLE 1C. ¹H NMR (300 MHz, DMSO-d₆) δ 11.29 (brs, 1H), 10.47 (brs, 1H), 10.05 (brs, 1H), 7.41 (s, 1H), 7.30 (d, 1H), 7.25 (s, 1H), 6.76 (d, 1H), 6.71 (s, 1H).

### EXAMPLE 6A

A suspension of 5-chloro-2,4-dimethoxyphenylamine (0.375 g), 1,2-difluoro-3-nitro-5-trifluoromethylbenzene (0.454 g) and potassium carbonate (0.276 g) in DMSO (5 mL) at 120° C. was stirred for 12 hours, poured to water and filtered. The filtrant was purified by flash chromatography on silica gel with 10-50% ethyl acetate/hexanes.

### EXAMPLE 6B

To a suspension of EXAMPLE 6B in methanol/dichloromethane (5/3 mL) was added Zn powder (200 mg) and hydrazinium formate (0.5 mL) (prepared by adding hydrazine monohydrate (5 mL) to 96% formic acid (4 mL) at 0-5° C.). The solution was stirred for 10 minutes, warmed to ambient temperature, stirred for 2 hours and concentrated. The concentrate was dissolved in dichloromethane, washed with water and brine and concentrated. The concentrate was again subjected to the preceeding reduction and work-up conditions, dissolved in dichloromethane, treated with 20% phosgene in toluene (0.5 mL), stirred at ambient temperature for 16 hours, quenched with water (0.2 mL) and concentrated. The concentrate was purified by preparative reverse phase HPLC (C-8, 20-100% acetonitrile/water/0.1% TFA).

### EXAMPLE 6C

This compound was made by substituting EXAMPLE 6B for EXAMPLE 1B in EXAMPLE 1C. ¹H NMR (300 MHz, DMSO-d₆) δ 11.59 (brs, 1H), 10.37 (brs, 1H), 10.03 (s, 1H), 7.35 (s, 1H), 7.27 (d, J=1 1.2 Hz, 1H), 7.15 (s, 1H), 6.66 (s, 1H).

### EXAMPLE 7A

This compound was made by substituting 4-fluoro-3-nitro-benzonitrile for 1,2-difluoro-3-nitro-5-trifluoromethylbenzene in EXAMPLE 6A.

### EXAMPLE 7B

This compound was made by substituting EXAMPLE 7A for EXAMPLE 6A in EXAMPLE 6B.

### EXAMPLE 7C

This compound was made by substituting EXAMPLE 7B for EXAMPLE 1B in EXAMPLE 1C. ¹H NMR (300 MHz, DMSO-d₆) δ 11.39 (brs, 1H), 10.43 (s, 1H), 10.04 (s, 1H), 7.41 (s, 1H), 7.40 (d, 1H), 7.30 (s, 1H), 6.73 (d, 1H), 6.71 (s, 1H).

### EXAMPLE 8A

This compound was made by substituting 3-fluoro-4-nitro-benzonitrile for 1,2-difluoro-3-nitro-5-trifluoromethyl-benzene in EXAMPLE 6A.

### EXAMPLE 8B

This compound was made by substituting EXAMPLE 8A for EXAMPLE 6A in EXAMPLE 6B.

### EXAMPLE 8C

This compound was made by substituting EXAMPLE 8B for EXAMPLE 1B in EXAMPLE 1C. ¹H NMR (300 MHz, DMSO-d₆) δ 11.52 (brs, 1H), 10.41 (s, 1H), 10.02 (s, 1H), 7.46 (dd, 1H), 7.29 (s, 1H), 7.16 (d, 1H), 6.99 (dd, 1H), 6.71 (s, 1H).

### EXAMPLE 9A

This compound was made by substituting 1,4-difluoro-2-nitro-benzene for 1,2-difluoro-3-nitro-5-trifluoromethyl-benzene in EXAMPLE 6A.

### EXAMPLE 9B

This compound was made by substituting EXAMPLE 9A for EXAMPLE 6A in EXAMPLE 6B.

### EXAMPLE 9C

This compound was made by substituting EXAMPLE 9B for EXAMPLE 1B in EXAMPLE 1C. ¹H NMR (300 MHz, DMSO-d₆) δ 11.08 (brs, 1H), 10.34 (brs, 1H), 9.93 (s, 1H), 7.23 (s, 1H), 6.86 (dd, 1H), 6.74 (ddd, 1H), 6.69 (s, 1H), 6.55 (dd, 1H).

### EXAMPLE 10A

This compound was made by substituting 4-fluoro-3-nitro-benzenesulfonamide for 1,2-difluoro-3-nitro-5-trifluoromethyl-benzene in EXAMPLE 6A.

### EXAMPLE 10B

This compound was made by substituting EXAMPLE 10A for EXAMPLE 6A in EXAMPLE 6B.

### EXAMPLE 10C

This compound was made by substituting EXAMPLE 10B for EXAMPLE 1B in EXAMPLE 1C. ¹H NMR (300 MHz, DMSO-d₆) δ 11.30 (s, 1H), 10.41 (s, 1H), 10.00 (s, 1H), 7.45 (m, 1H), 7.43 (s, 1H), 7.29 (s, 1H), 7.21 (s, 2H), 6.72 (d, 1H), 6.71 (s, 1H).

### EXAMPLE 11A

To a solution of 2-fluoro-5-nitro-benzoic acid (0.74 g) and 5-chloro-2,4-dimethoxy-phenylamine (0.75 g) in THF (30 mL) at 0-5° C. was added 1M LiHMDS in THF (12 mL). The solution was stirred for 18 hours at ambient temperature, quenched with 2M HCl (20 mL) and extracted with diethyl ether. The extract was dried (MgSO₄), filtered and concentrated. The concentrate was treated with ethanol/hexanes and filtered.

### EXAMPLE 11B

A solution of EXAMPLE 11A (0.352 g), DPPA (0.25 mL) and triethylamine (0.18 mL) at ambient temperature was stirred for 1 hour, heated at 100° C. for 3 days and concentrated. The concentrate was triturated with dichloromethane and filtered.

### EXAMPLE 11C

This compound was made by substituting EXAMPLE II B for EXAMPLE 1B in EXAMPLE 1C. ¹H NMR (300 MHz, DMSO-d₆) δ 11.53 (s, 1H), 10.47 (s, 1H), 10.10 (s, 1H), 7.95 (dd, 1H), 7.80 (d, 1H), 7.34 (s, 1H), 6.79 (d, 1H), 6.72 (s, 1H).

### EXAMPLE 12A

This compound was made by substituting 4-fluoro-3-chloro-benzonitrile for 2-fluoro-5-nitro-benzoic acid in EXAMPLE 11A.

### EXAMPLE 12B

This compound was made by substituting EXAMPLE 12A for EXAMPLE 11A in EXAMPLE 11B.

### EXAMPLE 12C

This compound was made by substituting EXAMPLE 12B for EXAMPLE 1B in EXAMPLE 1C. ¹H NMR (300 MHz, DMSO-d₆) δ 11.09 (brs, 1H), 10.36 (brs, 1H), 9.96 (s, 1H), 7.24 (s, 1H), 7.02 (d, 1H), 6.97 (dd, 1H), 6.69 (s, 1H), 6.57 (d, 1H).

### EXAMPLE 13A

This compound was made by substituting 4-fluoro-3-bromo-benzonitrile for 2-fluoro-5-nitro-benzoic acid in EXAMPLE 11A.

### EXAMPLE 13B

This compound was made by substituting EXAMPLE 13A for EXAMPLE 11A in EXAMPLE 11B.

### EXAMPLE 13C

This compound was made by substituting EXAMPLE 13B for EXAMPLE 1B in EXAMPLE 1C. ¹H NMR (300 MHz, DMSO-d₆) δ 11.09 (brs, 1H), 10.36 (brs, 1H), 9.94 (s, 1H), 7.24 (s, 1H), 7.14 (d, 1H), 7.09 (dd, 1H), 6.69 (s, 1H), 6.53 (d, 1H).

### EXAMPLE 14A

A suspension of EXAMPLE 5A (0.04 g), PdCl₂(PPh₃)₄ (6 mg), 2M Na₂CO₃ (0.1 mL) and phenylboronic acid (30 mg) in 7:3:2 DME/water/ethanol (2 mL) at 150° C. was stirred under microwaves for 10 minutes and filtered. The filtrant was washed with ethanol, and the filtrate was concentrated. The concentrate was purified by preparative reverse phase HPLC (Zorbax SB, C-18, 20-100% acetonitrile/water/0.1% TFA).

### EXAMPLE 14B

This compound was made by substituting EXAMPLE 14A for EXAMPLE 1B in EXAMPLE 1C. ¹H NMR (300 MHz, DMSO-d₆) δ 11.28 (s, 1H), 9.89 (s, 1H), 9.86 (s, 1H), 7.52 (m, 2H), 7.39-7.23 (m, 5H), 7.18 (s, 1H), 6.82 (d, 1H), 6.70 (s, 1H).

### EXAMPLE 15A

This compound was made by substituting EXAMPLE 4B for EXAMPLE 2B in EXAMPLE 5A.

### EXAMPLE 15B

This compound was made by substituting EXAMPLE 15A for EXAMPLE 1B in EXAMPLE 1C. ¹H NMR (500 MHz, DMSO-d₆) δ 10.90 (s, 1H), 10.41 (s, 1H), 9.93 (s, 1H), 7.34 (d, 1H), 7.02-6.90 (m, 3H), 6.71 (s, 1H), 6.58 (d, 1H).

### EXAMPLE 16A

This compound was made by substituting 2-phenylvinylboronic acid for phenylboronic acid in EXAMPLE 14A.

### EXAMPLE 16B

This compound was made by substituting EXAMPLE 16A for EXAMPLE 1B in EXAMPLE 1C. ¹H NMR (400 MHz, DMSO-d₆) δ 11.28 (s, 1H), 10.06 (s, 1H), 9.93 (s, 1H), 7.52 (s, 1H), 7.48 (d, 2H), 7.36-7.26 (m, 5H), 7.20 (tt, 1H), 7.06 (d, 1H), 6.79 (d, 1H), 6.63 (s, 1H).

### EXAMPLE 17A

This compound was made by substituting 2-phenylvinylboronic acid for phenylboronic acid and EXAMPLE 15A for EXAMPLE 5A in EXAMPLE 14A.

### EXAMPLE 17B

This compound was made by substituting EXAMPLE 17A for EXAMPLE 1B in EXAMPLE 1C. ¹H NMR (400 MHz, DMSO-d₆) δ 10.87 (s, 1H), 9.99 (s, 1H), 9.81 (s, 1H), 7.48 (d, 1H), 7.46 (s, 1H), 7.33 (d, 2H), 7.32 (d, 1H), 7.19 (tt, 1H), 7.06 (d, 1H), 7.02-6.90 (m, 4H), 6.61 (s, 1H), 6.60 (d, 1H).

### EXAMPLE 18A

This compound was made by substituting EXAMPLE 15A for EXAMPLE 5A in EXAMPLE 14A.

### EXAMPLE 18B

This compound was made by substituting EXAMPLE 18A for EXAMPLE 1B in EXAMPLE 1C. ¹H NMR (400 MHz, DMSO-d₆) δ 10.85 (s, 1H), 9.76 (brs, 2H), 7.52 (d, 2H), 7.35 (t, 2H), 7.23 (tt, 1H), 7.11 (s, 1H), 7.03-6.90 (m, 3H), 6.99 (s, 1H), 6.64 (d, 1H).

### EXAMPLE 19A

This compound was made by substituting 2-hydroxy-phenylboronic acid for phenylboronic acid and EXAMPLE 15A for EXAMPLE 5A in EXAMPLE 14A.

### EXAMPLE 19B

This compound was made by substituting EXAMPLE 19A for EXAMPLE 1B in EXAMPLE 1C. ¹H NMR (500 MHz, DMSO-d₆) δ 10.85 (s, 1H), 9.63 (s, 1H), 9.46 (s, 1H), 9.13 (s, 1H), 7.16 (dd, 1H), 7.09 (td, 1H), 7.01-6.95 (m, 3H), 6.93 (td, 1H), 6.86 (dd, 1H), 6.79 (td, 1H), 6.66 (d, 1H), 6.63 (s, 1H).

### EXAMPLE 20A

This compound was made by substituting 4-chloro-phenylboronic acid for phenylboronic acid and EXAMPLE 15A for EXAMPLE 5A in EXAMPLE 14A.

### EXAMPLE 20B

This compound was made by substituting EXAMPLE 20A for EXAMPLE 1B in EXAMPLE 1C. ¹H NMR (400 MHz, DMSO-d₆) δ 10.88 (brs, 1H), 9.92 (brs, 1H), 9.85 (brs, 1H) 7.56 (dt, 2H), 7.40 (dt, 2H), 7.16 (s, 1H), 7.03-6.95 (m, 2H), 6.93 (td, 1H), 6.69 (s, 1H), 6.64 (d, 1H).

### EXAMPLE 21A

This compound was made by substituting 2-chloro-phenylboronic acid for phenylboronic acid and EXAMPLE 15A for EXAMPLE 5A in EXAMPLE 14A.

### EXAMPLE 21B

This compound was made by substituting EXAMPLE 21A for EXAMPLE 1B in EXAMPLE 1C. ¹H NMR (400 MHz, DMSO-d₆) δ 10.85 (s, 1H), 9.94 (s, H), 9.86 (s, 1H), 7.58 (t, 1H), 7.48 (dt, 1H), 7.37 (t, 1H), 7.28 (ddd, 1H), 7.19 (s, 1H), 7.02-6.95 (m, 2H), 6.92 (td, 1H), 6.69 (s, 1H), 6.63 (d, 1H).

### EXAMPLE 22A

This compound was made by substituting 3-chloro-phenylboronic acid for phenylboronic acid and EXAMPLE 15A for EXAMPLE 5A in EXAMPLE 14A.

### EXAMPLE 22B

This compound was made by substituting EXAMPLE 22A for EXAMPLE 1B in EXAMPLE 1C. ¹H NMR (400 MHz, DMSO-d₆) δ 10.84 (brs, 1H), 9.77 (brs, 1H), 9.70 (s, 1H) 7.50-7.46 (m, 1H), 7.35-7.29 (m, 3H), 7.01-6.92 (m, 3H), 6.93 (s, 1H), 6.66 (s, 1H), 6.64 (d, 1H).

### EXAMPLE 23A

This compound was made by substituting 1-propenylboronic acid for phenylboronic acid and EXAMPLE 15A for EXAMPLE 5A in EXAMPLE 14A.

### EXAMPLE 23B

A suspension of EXAMPLE 23A (0.026 g) and 10% Pd/C (catalytic) in ethanol at ambient temperature was stirred under hydrogen for 16 hours, filtered through diatomaceous earth (Celite®) and concentrated.

### EXAMPLE 23C

This compound was made by substituting EXAMPLE 23B for EXAMPLE 1B in EXAMPLE 1C. ¹H NMR (400 MHz, DMSO-d₆) δ 10.80 (s, 1H), 9.38 (s, 1H), 9.31 (s, 1H),7.01-6.93 (m, 2H), 6.90 (td, 1H), 6.84 (s, 1H), 6.53 (d, 1H), 6.52 (s, 1H), 2.42 (t, 2H), 1.50 (sextet, 2 H), 0.88 (t, 3H).

### EXAMPLE 24A

This compound was made by substituting 4-methoxyphenyl-boronic acid for phenylboronic acid and EXAMPLE 13B for EXAMPLE 5A in EXAMPLE 14A.

### EXAMPLE 24B

This compound was made by substituting EXAMPLE 24A for EXAMPLE 1B in EXAMPLE 1C. ¹H NMR (300 MHz, DMSO-d₆) δ 10.95 (s, 1H), 10.34 (s, 1H), 9.92 (s, 1H), 9.43 (s, 1H), 7.39 (dt, 2H), 7.24 (s, 1H), 7.12 (sextet, 2H), 6.82 (dt, 2H), 6.71 (s, 1H), 6.61 (d, 1H).

### EXAMPLE 25A

This compound was made by substituting 4-methoxyphenyl-boronic acid for phenylboronic acid and EXAMPLE 13B for EXAMPLE 5A in EXAMPLE 14A.

### EXAMPLE 25B

This compound was made by substituting EXAMPLE 25A for EXAMPLE 1B in EXAMPLE 1C. ¹H NMR (300 MHz, DMSO-d₆) δ 11.03 (s, 1H), 10.36 (s, 1H), 9.94 (s, 1H), 7.60 (dt, 2H), 7.44 (t, 2H), 7.32 (tt, 1H), 7.26 (s, 1H), 7.25-7.20 (m, 2H), 6.72 (s, 1H), 6.67 (d, 1H).

The foregoing is meant to illustrate the invention but not to limit it. Variations and changes obvious to one skilled in the art are intended to be within the scope of the invention as defined in the claims.

We claim:

1. A compound having formula (I) formula (II) or formula (III) 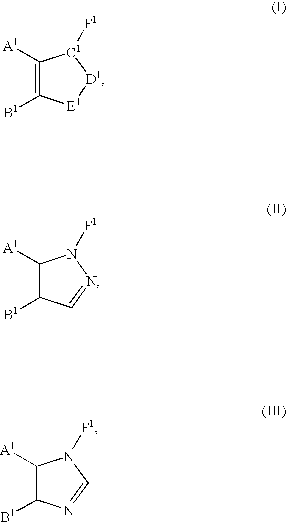
or a therapeutically acceptable salt thereof, wherein
A¹ and B¹ are together and are benzene;
C¹ is C(H) or N;
D¹ is CH₂, C(O), NH, O, S, S(O) or SO₂ and E is CH₂ or NH, or
D¹ is CH₂ or NH and E¹ is CH₂, C(O), NH, O, S, S(O) or SO₂;
F¹ is phenol-2-yl which is unfused or fused with F^{1A} and substituted at the 4-position by OH, NH₂, NHR¹, N(R¹)₂, C(O)NH₂, C(O)NHR¹, C(O)N(R¹)₂, NHC(O)R¹, NR¹C(O)R¹, SO₂NH₂, SO₂NHR¹, SO₂N(R¹)₂, NHSO₂R¹ or NR¹SO₂R¹;
F^{1A} is benzene, heteroarene, cycloalkane, cycloalkene, heterocycloalkane or heterocycloalkene;
R¹ is R², R³, R⁴ or R⁵;
R² is phenyl which is unfused or fused with benzene, heteroarene or R^{2A}; R^{2A} is cycloalkane, cycloalkene, heterocycloalkane or heterocycloalkene;
R³ is heteroaryl which is unfused or fused with benzene, heteroarene or R^{3A}; R^{3A} is cycloalkane, cycloalkene, heterocycloalkane or heterocycloalkene;
R⁴ is cycloalkyl, cycloalkenyl, heterocycloalkyl or heterocycloalkenyl, each of which is unfused or fused with benzene, heteroarene or R^{4A}; R^{4A} is cycloalkane, cycloalkene, heterocycloalkane or heterocycloalkene;
R⁵ is alkyl, alkenyl or alkynyl, each of which is unsubstituted or substituted with one or two or three of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHSO₂R⁶, NR⁶SO₂R⁶, NHC(O)OR⁶, NR⁶C(O)OR⁶, SO₂NH₂, SO₂NHR⁶, SO₂N(R⁶)₂, NHC(O)NH₂, NHC(O)NHR⁶, NHC(O)N(R⁶)₂, OH, (O), C(O)OH, CN, NH₂, CF₃, OCF₃, CF₂CF₃, F, Cl, Br or I;
R⁶ is R⁷, R⁸, R⁹ or R¹⁰;
R⁷ is phenyl which is unfused or fused with benzene, heteroarene or R^{7A}; R^{7A} is cycloalkane, cycloalkene, heterocycloalkane or heterocycloalkene;
R⁸ is heteroaryl which is unfused or fused with benzene, heteroarene or R^{8A}; R^{8A} is cycloalkane, cycloalkene, heterocycloalkane or heterocycloalkene;
R⁹ is cycloalkyl, cycloalkenyl, heterocycloalkyl or heterocycloalkenyl, each of which is unfused or fused with benzene, heteroarene or R^{9A}; R^{9A} is cycloalkane, cycloalkene, heterocycloalkane or heterocycloalkene;
R¹⁰ is alkyl, alkenyl or alkynyl, each of which is unsubstituted or substituted with one or two or three of independently selected OR¹¹, SR¹¹, S(O)R¹¹, SO₂R¹¹, NH₂, NHR¹¹, N(R¹¹)₂, C(O)R₁₁, C(O)NH₂, C(O)NHR¹¹, C(O)N(R¹¹)₂, NHC(O)R¹¹, NR¹¹C(O)R¹¹, NHSO₂R¹¹, NR¹¹SO₂R¹¹, NHC(O)OR¹¹, NR¹¹C(O)OR¹¹, SO₂NH₂, SO₂NHR¹¹, SO₂N(R¹¹)₂, NHC(O)NH₂, NHC(O)NHR¹¹, NHC(O)N(R¹¹)₂, NR¹¹C(O)N(R¹¹)₂, OH, (O), C(O)H, C(O)OH, CN, CF₃, OCF₃, CF₂CF₃, F, Cl, Br or I;
R¹¹ is alkyl, alkenyl, alkynyl, phenyl, naphthyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl or 1,2,3-triazolyl;
wherein the benzene represented by A¹ and B¹ together and the moieties represented by F¹ and F^{1A} are independently unsubstituted or substituted or further substituted with one or two or three or four of independently selected R¹², OR¹², SR¹², S(O)R¹², SO₂R¹², NH₂, NHR¹², N(R¹²)₂, C(O)R¹², C(O)OR¹², C(O)NH₂, C(O)NH R¹², C(O)N(R¹²) NHC(O)R¹², NR¹²C(O)R¹², NHSO₂R¹², NR¹²SO₂R¹², NHC(O)OR¹², NR¹²C(O)OR¹², SO₂NH₂, SO₂NHR¹², SO₂N(R¹²)₂, NHC(O)NH₂, NHC(O)NHR¹², NHC(O)N(R¹²)₂, NR¹²C(O)N(R¹²)₂, OH, (O), C(O)H, C(O)OH, NO₂, CN, CF₃, OCF₃, CF₂CF₃, F, Cl, Br or I;
wherein R¹² is R¹³,R¹⁴,R¹⁵ or R¹⁶;
R¹³ is phenyl which is unfused or fused with benzene, heteroarene or R^{13A}; R^{13A} is cycloalkane, cycloalkene, heterocycloalkane or heterocycloalkene;
R¹⁴ is heteroaryl which is unfused or fused with benzene, heteroarene or R^{14A}; R^{14A} is cycloalkane, cycloalkene, heterocycloalkane or heterocycloalkene;
R¹⁵ is cycloalkyl, cycloalkenyl, heterocycloalkyl or heterocycloalkenyl, each of which is unfused or fused with benzene, heteroarene or R^{5A}; R^{15A} is cycloalkane, cycloalkene, heterocycloalkane or heterocycloalkene;
R¹⁶ is alkyl, alkenyl or alkynyl, each of which is unsubstituted or substituted with one or two or three of independently selected R¹⁷, OR¹⁷, SR¹⁷, S(O)R¹⁷, SO₂R¹⁷, NH₂, NHR¹⁷, N(R¹⁷)₂, C(O)R¹⁷, C(O)NH₂, C(O)NHR¹⁷, C(O)N(R¹⁷)₂, NHC(O)R¹⁷, NR¹⁷C(O)R¹⁷, NHSO₂R¹⁷, NR¹⁷SO₂R¹⁷, NHC(O)OR¹⁷, NR¹⁷C(O)OR¹⁷, SO₂NH₂, SO₂NHR¹⁷, SO₂N(R¹⁷)₂, NHC(O)NH₂, NHC(O)NHR¹⁷, NHC(O)N(R¹⁷)₂, NR¹⁷C(O)N(R¹⁷)₂, OH, (O), C(O)H, C(O)OH, CN, CF₃, CF₂CF₃, OCF₃, F, Cl, Br or I;
R¹⁷ is R¹⁸, R¹⁹, R²⁰ or R²¹;
R¹⁸ is phenyl which is unfused or fused with benzene, heteroarene or R^{8A}; R^{18A} is cycloalkane, cycloalkene, heterocycloalkane or heterocycloalkene;
R¹⁹is heteroaryl which is unfused or fused with benzene, heteroarene or R^{19A} R^{19A} is cycloalkane, cycloalkene, heterocycloalkane or heterocycloalkene;
R²⁰ is cycloalkyl, cycloalkenyl, heterocycloalkyl or heterocycloalkenyl, each of which is unfused or fused with benzene, heteroarene or R^{20A}; R^{20A} is cycloalkane, cycloalkene, heterocycloalkane or heterocycloalkene;
R²¹ is alkyl, alkenyl or alkynyl, each of which is unsubstituted or substituted with one or two or three of independently selected R²², OR²², SR²², S(O)R²², SO₂R²², NH₂, NHR²², N(R²²)₂, C(O)R²², C(O)NH₂, C(O)NHR²², C(O)N(R²²)₂, NHC(O)R²², NR²²C(O)R²², NHSO₂R²², NR²²SO₂R²², NHC(O)OR²², NR¹⁷C(O)OR²², SO₂NH₂, SO₂NHR²², SO₂N(R²²)₂, NHC(O)NH₂, NHC(O)NHR₂, NHC(O)N(R²²)₂, NR²²C(O)N(R²²)₂, OH, (O), C(O)H, C(O)OH, CN, CF₃, OCF₃, CF₂CF₃, F, Cl, Br or I;
R²² is R²³, R²⁴, R²⁵ or R²⁶;
R²³ is phenyl which is unfused or fused with benzene, heteroarene or R^{23A}; R^{23A} is cycloalkane, cycloalkene, heterocycloalkane or heterocycloalkene;
R²⁴ is heteroaryl which is unfused or fused with benzene, heteroarene or R^{24A}; R^{24A} is cycloalkane, cycloalkene, heterocycloalkane or heterocycloalkene;
R²⁵ is cycloalkyl, cycloalkenyl, heterocycloalkyl or heterocycloalkenyl, each of which is unfused or fused with benzene, heteroarene or R^{25A}; R^{25A} is cycloalkane, cycloalkene, heterocycloalkane or heterocycloalkene;
R²⁶ is alkyl, alkenyl or alkynyl;
wherein the moieties represented by R¹³, R¹⁴, R¹⁵, R¹⁸, R¹⁹ and R²⁰ are independently unsubstituted or substituted or further substituted with one or two or three or four of independently selected R²⁷, OR²⁷, SR²⁷, S(O)R²⁷, SO₂R²⁷, NH₂, NHR²⁷, N(R²⁷)₂, C(O)R²⁷, C(O)OR²⁷, C(O)NH₂, C(O)NHR²⁷, C(O)N(R²⁷)₂, NHC(O)R²⁷, NR²⁷C(O)R²⁷, NHSO₂R²⁷, NR²⁷SO₂R , NHC(O)OR²⁷, NR²⁷C(O)OR²⁷, SO₂NH₂, SO₂NR²⁷, SO₂N(R²⁷)₂, NHC(O)NH₂, NHC(O)NHR²⁷, NHC(O)N(R²⁷)₂, NR²⁷C(O)N(R²⁷)₂, C(N)NH₂, C(N)NHR²⁷, C(N)N(R²⁷)₂, NHC(N)NH₂, NHC(N)NHR²⁷, NHC(N)N(R²⁷)₂, OH, (O), C(O)H, C(O)OH, NO₂, CN, CF₃, OCF₃, CF₂CF₃, F, Cl, Br or I;
R²⁷ is R²⁸, R²⁹, R³⁰ or R³¹;
R²⁸ is phenyl which is unfused or fused with benzene, heteroarene or R^{27A}; R^{27A} is cycloalkane, cycloalkene, heterocycloalkane or heterocycloalkene;
R²⁹ is heteroaryl which is unfused or fused with benzene, heteroarene or R^{28A}; R^{28A} is cycloalkane, cycloalkene, heterocycloalkane or heterocycloalkene;
R³⁰ is cycloalkyl, cycloalkenyl, heterocycloalkyl or heterocycloalkenyl, each of which is unfused or fused with one or two of independently selected benzene, heteroarene or R^{29A}; R^{29A} is cycloalkane, cycloalkene, heterocycloalkane or heterocycloalkene;
R³¹ is alkyl, alkenyl or alkynyl;
wherein the moieties represented by R²⁸, R²⁹ and R³⁰ are unsubstituted or substituted with OH, (O), C(O)H, C(O)OH, NO₂, CN, CF₃, OCF₃, CF₂CF₃, F, Cl, Br or I.

2. A composition comprising an excipient and a therapeutically effective amount of the compound having formula (I), formula (II) or formula (III).

3. A method of treating acute lymphocytic leukemia, breast cancer, cervical cancer, chronic myelogenous leukemia, colon cancer, lung cancer, melanoma, ovarian cancer, pancreatic cancer, prostate cancer, renal carcinoma and squamous cell carcinoma, said methods comprising administering to the patient a therapeutically effective amount of a compound having formula (I), formula (II) or formula (III).
